(19) United States
(12) Patent Application Publication
(10) Pub. No.: US 2025/0229131 A1
(43) Pub. Date: Jul. 17, 2025

(54) WEARABLE DEVICE CONTROL METHOD AND WEARABLE DEVICE FOR PROVIDING NOTIFICATION TO USER

(71) Applicant: SAMSUNG ELECTRONICS CO., LTD., Suwon-si (KR)

(72) Inventors: Sugyeong KIM, Suwon-si (KR); Seungjoon LEE, Suwon-si (KR); Hyunkyung SONG, Suwon-si (KR); Hoon HAN, Suwon-si (KR)

(21) Appl. No.: 19/170,715

(22) Filed: Apr. 4, 2025

Related U.S. Application Data
(63) Continuation of application No. PCT/KR2023/014569, filed on Sep. 22, 2023.

(30) Foreign Application Priority Data
Oct. 4, 2022 (KR) 10-2022-0126553

(51) Int. Cl. A63B 24/00; G16H 40/67

(52) U.S. Cl. CPC A63B 24/0062; A63B 24/0075; G16H 40/67; A63B 2024/0065; A63B 2024/0068

(57) ABSTRACT

A wearable device control method may include: receiving condition information including a wearable device search condition from a user terminal; receiving device information of each of multiple wearable from each of the wearable devices; selecting at least one wearable device for a user from among the wearable devices on the basis of each piece of received state information and the received condition information; and transmitting, to the selected wearable device, a control signal causing a notification indicating that the selected wearable device matches the wearable device search condition to be provided to a user.

### CROSS-REFERENCE TO RELATED APPLICATIONS

This application is a continuation application of International Application No. PCT/KR2023/014569 designating the United States, filed on Sep. 22, 2023, in the Korean Intellectual Property Receiving Office and claiming priority to Korean Patent Application No. 10-2022-0126553, filed on Oct. 4, 2022, the disclosures of which are all hereby incorporated by reference herein in their entireties.

### BACKGROUND

### Technical Field

Certain example embodiments may relate to a wearable device control method and/or a wearable device for providing a notification to a user.

### Background Art

A wearable device may apply an external force to a user to help the user perform exercise. For example, the wearable device may provide the user with an assistance force to assist a motion of the user and/or may provide the user with a resistance force to act as resistance to a motion of the user.

### SUMMARY

In a situation in which there is a plurality of wearable devices, a user may not be aware of the size and charge state of each of the plurality of wearable devices. The user may have difficulty finding an optimal wearable device that fits the size of the user and has the charging capacity to match the amount of exercise the user is expected to perform.

A wearable device control method performed by a server according to an example embodiment may include an operation of receiving, from a user terminal, condition information including a wearable device search condition. The wearable device control method may include an operation of receiving device information of each of a plurality of wearable devices from each of the wearable devices. The wearable device control method may include an operation of selecting at least one wearable device for a user among the wearable devices based on the received state information and the received condition information. The wearable device control method may include an operation of transmitting a control signal to the selected wearable device to provide a user with a notification indicating that the selected wearable device matches the wearable device search condition.

A wearable device according to an example embodiment may include a communication module, comprising communication circuitry, configured to communicate with a server, a driving module including an actuator, comprising a motor and/or circuitry, configured to generate a torque, and at least one processor, comprising processing circuitry, individually and/or collectively configured to: receive, from the server through the communication module, a control signal to provide a user with a notification indicating that the wearable device matches a wearable device search condition of the user and perform, based on the received control signal, at least one of driving the actuator or providing visual feedback.

In an example embodiment, when a user selects to search for a device using a user terminal in a situation in which wearable devices of the same shape are located, one or more wearable devices selected as appropriate for the user may output a notification (e.g., lighting or a motion), thereby allowing the user to intuitively determine which wearable device is appropriate for the user among the plurality of wearable devices.

### BRIEF DESCRIPTION OF DRAWINGS

FIG. 1A is a diagram illustrating an overview of a wearable device worn on a body of a user, according to an example embodiment.

FIG. 1B is a diagram illustrating an example of a system including a wearable device, according to an example embodiment.

FIG. 2A is a rear schematic view of a wearable device according to an example embodiment.

FIG. 2B is a left side view of a wearable device according to an example embodiment.

FIGS. 3A and 3B are block diagrams illustrating examples of a configuration of a wearable device, according to an example embodiment.

FIG. 4 is a flowchart illustrating an example of operations of a user terminal, a server, and wearable devices, according to an example embodiment.

FIG. 5 is a flowchart illustrating an example of an operation of a wearable device matching a user terminal, a server, and a search condition, according to an example embodiment.

FIG. 6 is a diagram illustrating an example of a motion of a wearable device matching a search condition related to an exercise, according to an example embodiment.

FIG. 7 is a diagram illustrating an example of a motion of a wearable device matching a search condition related to a charge rate, according to an example embodiment.

FIGS. 8 and 9 are diagrams illustrating a motion of a wearable device matching a search condition related to a size, according to an example embodiment.

FIG. 10 is a diagram illustrating an example of a wearable device performing different motions depending on the number of satisfied standards, according to an example embodiment.

FIG. 11 is a diagram illustrating an example of performing different motions depending on the state of a wearable device, according to an example embodiment.

FIG. 12 is a flowchart illustrating an operation of stopping a notification from another wearable device when a user terminal is connected to a wearable device, according to an example embodiment.

FIGS. 13 and 14 are diagrams illustrating examples of operations of a server and a user terminal when the same wearable device is recommended to a plurality of users, according to an example embodiment.

FIGS. 15, 16, and 17 are diagrams illustrating examples of operations of a user terminal, a server, and a wearable device when different wearable devices are recommended to a plurality of users, according to an example embodiment.

FIGS. 18A and 18B are diagrams illustrating examples of operations of a user terminal, a server, and an administrator terminal when there is a wearable device usage and exercise reservation, according to an example embodiment.

FIGS. 18C and 18D are diagrams illustrating examples of operations of a user terminal, a server, and a wearable device when there is a wearable device usage and exercise reservation, according to an example embodiment.

FIG. 19 is a block diagram illustrating an example of a server according to an example embodiment.

FIG. 20 is a block diagram illustrating an example of a wearable device according to an example embodiment.

### DETAILED DESCRIPTION

The following detailed structural or functional description is provided as an example only and various alterations and modifications may be made to embodiments. Accordingly, the embodiments are not construed as limited to the disclosure and should be understood to include all changes, equivalents, and replacements within the idea and the technical scope of the disclosure.

Although terms, such as first, second, and the like are used to describe various components, the components are not limited to the terms. These terms should be used only to distinguish one component from another component. For example, a first component may be referred to as a second component, and similarly the second component may also be referred to as the first component.

It should be noted that if one component is described as being “connected”, “coupled”, or “joined” to another component, at least a third component may be “connected”, “coupled”, and “joined” between the first and second components, although the first component may be directly connected, coupled, or joined to the second component. Thus, “connected” as used herein covers direct and indirect connections.

The singular forms “a”, “an”, and “the” are intended to include the plural forms as well, unless the context clearly indicates otherwise. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof.

Unless otherwise defined, all terms, including technical and scientific terms, used herein have the same meaning as commonly understood by one of ordinary skill in the art to which this disclosure pertains. Terms, such as those defined in commonly used dictionaries, are to be interpreted as having a meaning that is consistent with their meaning in the context of the relevant art, and are not to be interpreted in an idealized or overly formal sense unless expressly so defined herein.

Hereinafter, the embodiments will be described in detail with reference to the accompanying drawings. When describing the embodiments with reference to the accompanying drawings, like reference numerals refer to like elements and any repeated description related thereto will be omitted.

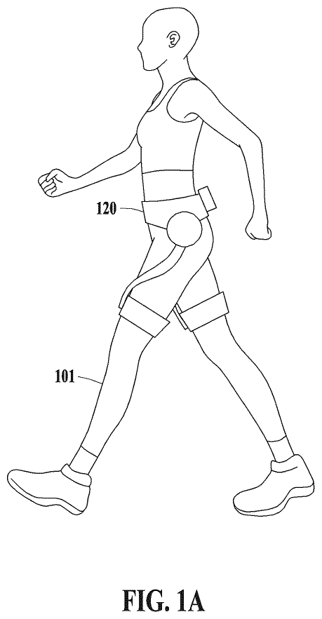

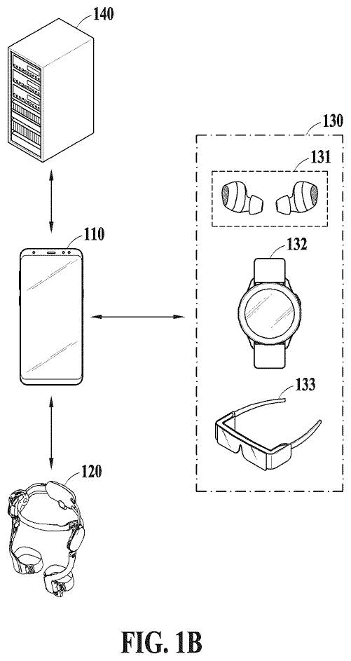

FIG. 1A is a diagram illustrating an overview of a wearable device worn on a body of a user, according to an embodiment.

Referring to FIG. 1A, a wearable device 120 may be a device worn on a body of a user 101 to assist the user 101 in walking, exercising, and/or working. In an embodiment, the term “wearable device” may be replaced with “wearable robot,” or “walking assistance device”. The user 101 may be a human or an animal, but is not limited thereto. The wearable device 120 may be worn on a body (e.g., a lower body (the legs, ankles, knees, etc.), an upper body (the torso, arms, wrists, etc.), or the waist) 10 of the user 101 to provide an external force such as an assistance force and/or a resistance force to a body motion of the user 101. The assistance force may be a force applied in the same direction as the body motion direction of the user 101, and the resistance force may be a force applied in a direction opposite to the body motion direction of the user 101. The term “resistance force” may also be referred to as “exercise load”.

When the wearable device 120 performs a walking assist function to assist the user 101 in walking, the wearable device 120 may assist a portion or entirety of a leg of the user 101 by providing an assistance force to the body of the user 101, thereby assisting the user 101 in walking. The wearable device 120 may enable the user 101 to walk independently or to walk for a long time by providing a force required for the user 101 to walk, thereby extending the walking ability of the user 101. The wearable device 120 may help in improving an abnormal walking habit or walking posture of a walker.

When the wearable device 120 performs an exercise function to enhance the exercise effect of the user 101, the wearable device 120 may hinder a body motion of the user 101 or provide resistance to a body motion of the user 101 by providing a resistance force to the body of the user 101. When the wearable device 120 is, for example, a hip-type wearable device, the wearable device 120 may provide an exercise load to a body motion of the user 101 while being worn on the legs, thereby enhancing the exercise effect of the user 101. The user 101 may perform a walking motion while wearing the wearable device 120 for exercise. In this case, the wearable device 120 may apply a resistance force to the leg motion during the walking motion of the user 101.

In various embodiments of the present disclosure, an example of a hip-type wearable device 120 that is worn on the waist and legs is described for ease of description. However, as described above, the wearable device 120 may be worn on another body part (e.g., the upper arms, lower arms, hands, calves, and feet) other than the waist and legs (particularly, the thighs), and the shape and configuration of the wearable device 120 may vary depending on the body part on which the wearable device 120 is worn.

FIG. 1B is a diagram illustrating an example of a system including a wearable device, according to an embodiment.

Referring to FIG. 1B, a user terminal 110 may communicate with the wearable device 120 and remotely control the wearable device 120. The user terminal 110 may be various types of electronic devices. The user terminal 110 may include, for example, a portable communication device (e.g., a smartphone), a computer device, a portable multimedia device, or a home appliance, but is not limited thereto.

According to an embodiment, the user terminal 110 and/or the wearable device 120 may be connected to another wearable device 130. For example, the wearable device 120, the user terminal 110, and the other wearable device 120 may be connected to one another through wireless communication (e.g., Bluetooth communication). The other wearable device 130 may include, for example, wireless earphones 131, a smart watch 132, or smart glasses 133, but is not limited thereto.

In an embodiment, the smart watch 132 may obtain biometric information (e.g., heart rate information) of the user and transmit the obtained biometric information to the user terminal 110.

In an embodiment, the user terminal 110 may be connected to a server 140 using short-range wireless communication (e.g., wireless-fidelity (Wi-Fi)) or mobile communication (e.g., fourth generation (4G), 5G, etc.).

In an embodiment, the user terminal 110 may receive profile information of the user 101 from the user 101. The profile information may include, for example, at least one of the age, gender, height, weight, or body mass index (BMI), or a combination thereof. The user terminal 110 may transmit the profile information of the user 101 to the server 140.

The user terminal 110 and/or the wearable device 120 may request the user to perform one or more target motions to determine (or check) the exercise ability of the user. The one or more target motions may include, for example, a knee lift, a backward leg stretch, etc. The knee lift may be a motion that starts from a position of the user 101 standing straight with two feet on the ground and returns to the standing position after lifting a leg backward as much as possible without bending at the waist. The backward leg stretch may be a motion that starts from a position of the user 101 standing straight with the hands on the wall and returns to the standing position after lifting a leg backward as much as possible without bending at the waist.

The wearable device 120 may obtain motion information of the user performing a target motion using a sensor (e.g., an inertial measurement unit (IMU)) and transmit the obtained motion information to the user terminal 110. The user terminal 110 may transmit the obtained motion information to the server 140.

The server 140 may determine a target amount of exercise of the user 101 for each of the exercise types (e.g., strength training, balance exercise, and aerobic exercise) through the profile information and motion information received from the user terminal 110. The server 140 may transmit the target amount of exercise for each exercise type to the user terminal 110. As described below, the user terminal 110 may execute a fitness application and display an execution screen including the target amount of exercise for each exercise type on the display.

In an embodiment, the server 140 may include a database in which information about a plurality of exercise programs to be provided to the user through the wearable device 120 is stored. For example, the server 140 may manage a user account of the user of the user terminal 110 or the wearable device 120. The server 140 may store and manage a workout program performed by the user and a result of performance with respect to the workout program in link with the user account.

In an embodiment, the user terminal 110 and/or the server 140 may provide the user with various exercise programs to achieve an exercise goal in various exercise environments desired by the user. The exercise goal may include, for example, at least one of muscle strength improvement, physical strength improvement, cardiovascular endurance improvement, core stability improvement, flexibility improvement, or symmetry improvement, or a combination thereof.

In an embodiment, the user terminal 110 and/or the server 140 may recommend exercise programs to the user to achieve the exercise goal of the user. Each exercise program may include one or more exercise modes. For example, each exercise mode may be about a body motion to achieve a predetermined exercise goal. For example, running may be an exercise mode for improving the cardiovascular endurance of the user. For example, a lunge may be an exercise mode for improving the core stability of the user. A combination of a plurality of exercise modes forming each exercise program may vary according to the exercise goal of the user. The user terminal 110 may provide the user with various exercise programs according to the combination of the plurality of exercise modes, even for the same exercise goal.

In an embodiment, the plurality of exercise modes may be stored in the user terminal 110 or the server 140 as a database. The user terminal 110 or the server 140 may generate the plurality of exercise programs based on a variety of information about the user and recommend a target exercise program among the plurality of exercise programs to the user in consideration of the exercise goal or an exercise performance state of the user. For example, the user terminal 110 or the server 140 may determine the target exercise program to recommend to the user based on at least one of the exercise goal, an exercise history, or an exercise performance result of the user. Accordingly, a new exercise program may be recommended to the user even if the user performs an exercise every day under the same exercise goal, and the user may feel like performing a different exercise from the previous exercise by performing the new exercise program.

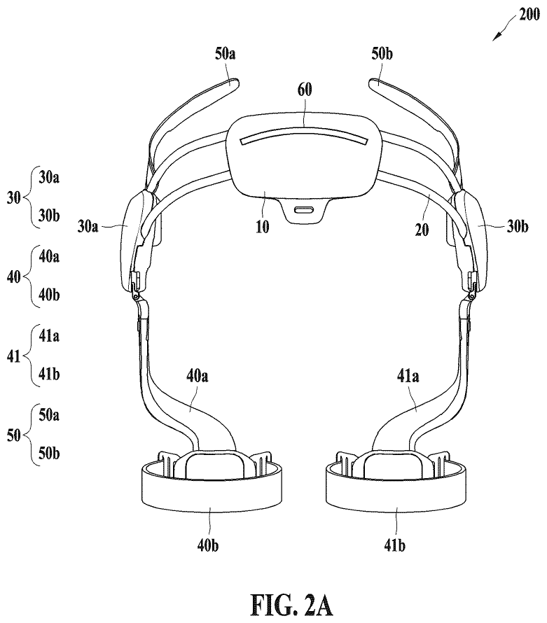

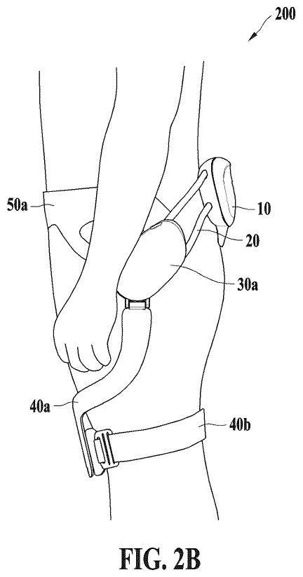

FIG. 2A is a rear schematic view of a wearable device according to an embodiment. FIG. 2B is a left side view of a wearable device according to an embodiment.

Referring to FIG. 2A, a wearable device 200 (e.g., the wearable device 120) according to an embodiment may include a base body 10, a base frame 20, driving modules 30a and 30b, a pair of arms 40a and 40b, and a main belt 50.

The base body 10 may be positioned on a lumbar region (an area of the lower back) of a user while the user is wearing the wearable device 200. The base body 10 may be mounted on, directly or indirectly, the lumbar region of the user to provide a cushioning feeling to the lower back of the user and may support the lower back of the user. The base body 10 may be hung on the hip region (an area of the hips) of the user to prevent or reduce chances of the wearable device 200 from being separated downward due to gravity while the user is wearing the wearable device 200. The base body 10 may distribute a portion of the weight of the wearable device 200 to the lower back of the user while the user is wearing the wearable device 200. The base body 10 may be connected to the base frame 20. Base frame connecting elements (not shown) to be connected to the base frame 20 may be provided at both end portions of the base body 10.

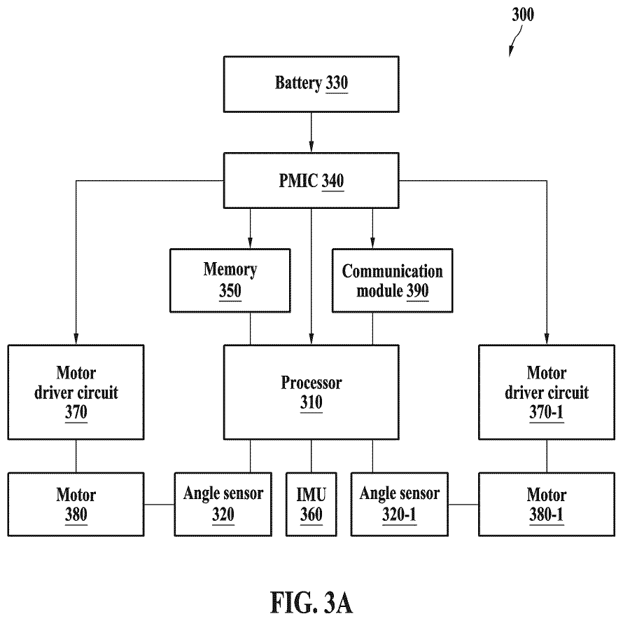

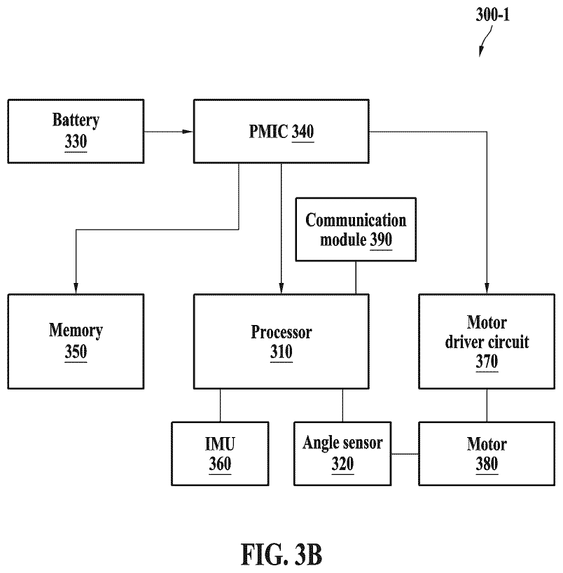

The base body 10 may include a lighting unit 60. The lighting unit 60 may include a plurality of light sources (e.g., light-emitting diodes (LEDs)). The lighting unit 60 may emit light by control of a processor (e.g., a processor 310 of FIGS. 3A and 3B). According to an embodiment, the processor may control the lighting unit 60 to provide (or output) visual feedback corresponding to the state of the wearable device 200 to the user through the lighting unit 60.

The base frame 20 may extend from both end portions of the base body 10. The lumbar region of the user may be accommodated inside the base frame 20. The base frame 20 may include at least one rigid body beam. Each beam may be in a curved shape having a preset curvature to enclose the lumbar region of the user. The main belt 50 may be connected to an end portion of the base frame 20. A driving module 30 may be mounted on, directly or indirectly, the base frame 20. The base frame 20 may include a connector (not shown) for mounting the driving module 30 thereon.

The driving module 30 may include a first driving module 30a positioned on the left side of the user while the user is wearing the wearable device 200 and a second driving module 30b positioned on the right side of the user while the user is wearing the wearable device 200.

The first driving module 30a may include a first actuator (not shown) and a first joint member (not shown), and the first driving module 30a may include a second actuator (not shown) and a second joint member (not shown). The first actuator may provide power to be transmitted to the first joint member, and the second actuator may provide power to be transmitted to the second joint member. The first actuator and the second actuator may each include a motor configured to generate power by receiving power from a battery (e.g., a battery 330 of FIGS. 3A and 3B). When the motor is driven as the power is supplied to the motor, the motor may provide an assistance force for assisting a body motion of the user or a resistance force for hindering a body motion of the user.

The first joint member may be rotated by the power received from the first actuator. A first encoder for measuring a rotation angle of the first joint member may be disposed on one side of the first joint member. The second joint member may be rotated by the power received from the second actuator. A second encoder for measuring a rotation angle of the second joint member may be disposed on one side of the second joint member.

The driving module 30 may be mounted and fixed to the base frame 20. The driving module 30 may transmit power to a pair of arms 40 and 41. The driving module 30 may be positioned on the side of a femoral region of a user, the side of the lower back of the user, or between the side of the femoral region and the side of the lower back, while the user is wearing the wearable device 200.

The pair of arms 40 and 41 may receive the power from the driving module 30 and pressurize a leg of the user in a direction that assists the movement of the femoral region, a knee, a calf, and the like of the user.

The pair of arms 40 may include a first arm 40 connected to the first driving module 30a and a second arm 41 connected to the second driving module 30b.

The first arm 40 may include a first support unit 40b that wraps around the left leg of the user and a first connection frame 40a that connects the first support unit 40b to the first driving module 30a.

The second arm 41 may include a second support unit 41b that wraps around the right leg of the user and a second connection frame 41a that connects the second support unit 41b to the second driving module 30b.

The connection frame 40a, 41a may support a leg (e.g., a thigh) of the user when the wearable device 200 is worn on the leg of the user. The connection frame 40a, 41a may also be referred to as a leg support frame. For example, the connection frame 40a, 41a may transmit power generated by the driving module 30a, 30b to the thigh of the user, and the power may act as an external force to be applied to the movement of the leg of the user. As an one end of the connection frame 40a, 41a rotates by being connected to the first and second joint members and the other end of the connection frame 40a, 41a is connected to the support unit 40b, 41b, the connection frame 40a, 41a may transmit the power generated by the driving module 30a, 30b to the thigh of the user while supporting the thigh of the user. For example, the connection frame 40a, 41a may push or pull the thigh of the user. The connection frame 40a, 41a may extend in the longitudinal direction of the thigh of the user. The connection frame 40a, 41a may be bent to surround at least a portion of the circumference of the thigh of the user.

The first driving module 30a may cause the first connection frame 40a to move (or rotate) in the forward direction or backward direction of the wearable device 200 by generating a torque. The second driving module 30b may cause the second connection frame 41a to move (or rotate) in the forward direction or backward direction of the wearable device 200 by generating a torque. The forward direction may be a direction corresponding to the front direction of the user or the flexion motion of a leg, and the backward direction may be a direction corresponding to the rear direction of the user or the extension motion of a leg.

The main belt 50 may be connected to the base frame 20. The main belt 50 may include a first main belt 50a configured to enclose the left abdomen of the user while the user is wearing the wearable device 200, and a second main belt 50b configured to enclose the right abdomen of the user while the user is wearing the wearable device 200. The first main belt 50a may be formed in a shape having a longer length than the second main belt 50b, but is not limited thereto, and the first main belt 50a may be formed in a shape having the same length as or a shorter length than the second main belt 50b. The first main belt 50a and the second main belt 50b may be connected to both end portions of the base frame 20, respectively. The main belt 50 may be bent in a direction to surround the abdomen of the user when the body of the user is inserted in such a direction that it is accommodated in the wearable device 200. The first main belt 50a and the second main belt 50b may be connected to each other while the user is wearing the wearable device 200. The main belt 50 may distribute a portion of the weight of the wearable device 200 to the abdomen of the user while the user is wearing the wearable device 200.

Referring to FIG. 2B, the base body 10 may be mounted on, directly or indirectly, the back of the lumbar region of the user and be hung on the hip region of the user, thereby supporting a portion of the weight of the wearable device 200. The first driving module 30a may be arranged on the left lumbar region of the user. The base frame 20 may extend from the end portion of the base body 10 and be inclined in a direction toward the first driving module 30a. The first main belt 50a mounted on, directly or indirectly, the base frame 20 may surround the left abdomen of the user. The first arm 40 may be mounted on, directly or indirectly, the leg of the user and may receive the power from the first driving module 30a.

FIGS. 3A and 3B are block diagrams illustrating examples of a configuration of a wearable device, according to an embodiment.

According to an embodiment, a wearable device 300 (e.g., the wearable device 120 and the wearable device 200) may include a processor 310, angle sensors 320 and 320-1, a battery 330, a power management integrated circuit (PMIC) 340, a memory 350, an IMU 360, motor driver circuits 370 and 370-1, motors 380 and 380-1, and a communication module 390.

Although the plurality of angle sensors 320 and 320-1, the plurality of motor driver circuits 370 and 370-1, and the plurality of motors 380 and 380-1 are shown in FIG. 3, which is merely an example, the wearable device 300-1 in the example shown in FIG. 3B may include a single angle sensor 320, a single motor driver circuit 370, and a single motor 380. Also, according to the implementation, the wearable devices 300 and 300-1 may include a plurality of processors. The number of motor driver circuits, the number of motors, or the number of processors may vary depending on a body part on which the wearable devices 300 and 300-1 are worn.

The angle sensor 320, the motor driver circuit 370, and the motor 380 may be included in the first driving module 30a of FIG. 2A, and the angle sensor 320-1, the motor driver circuit 370-1, and the motor 380-1 may be included in the second driving module 30b.

The angle sensor 320 may measure or sense an angle of a first joint (e.g., a left hip joint) of a user. The angle sensor 320 may transmit, to the processor 310, first angle information obtained by measuring the angle of the first joint. For example, the angle sensor 320 may measure a left hip joint angle of the user and may transmit, to the processor 310, angle information obtained by measuring the left hip joint angle.

The angle sensor 320-1 may measure an angle of a second joint (e.g., a right hip joint) of the user and may transmit, to the processor 310, second angle information obtained by measuring angle of the second joint.

According to an embodiment, the angle sensors 320 and 320-1 may additionally measure knee angles and ankle angles of the user depending on the positions of the angle sensors 320 and 320-1.

The angle sensors 320 and 320-1 may be, for example, the first encoder and the second encoder, respectively, described with reference to FIG. 2A.

According to an embodiment, the wearable devices 300 and 300-1 may include a potentiometer. The potentiometer may sense an R-axis joint angle, an L-axis joint angle, an R-axis joint angular velocity, and an L-axis joint angular velocity according to a gait motion of the user. In this example, the R and L axes may be reference axes for the right leg and the left leg of the user, respectively. For example, the R and L axes may be set to be vertical to the ground and set such that a front side of a body of a person has a negative value and a rear side of the body has a positive value.

The PMIC 340 may charge the battery 330 using power supplied from an external power source. For example, the external power source and the wearable devices 300 and 300-1 may be connected through a cable (e.g., a universal serial bus (USB) cable, etc.). The PMIC 340 may receive power from the external power source through the cable, and charge the battery 330 using the received power. According to an embodiment, the PMIC 340 may charge the battery 330 through a wireless charging method.

The PMIC 340 may transmit the power stored in the battery 330 to a component in the wearable device 300 or 300-1. For example, the PMIC 340 may adjust the power stored in the battery 330 to a voltage or a current level suitable for a component (e.g., the processor 310, the angle sensors 320 and 320-1, the memory 350, the IMU 360, and the motors 380 and 380-1) in the wearable device 300. The PMIC 340 may include, for example, a converter (e.g., a direct current (DC)-DC converter) or a regulator (e.g., a low-dropout (LDO) regulator or a switching regulator) configured to perform the adjustment described above.

The PMIC 340 may determine state information (e.g., a state of charge, a state of health, an overvoltage, a low voltage, an overcurrent, an overcharge, an overdischarge, an overheating, a short circuit, or a swelling) of the battery 330, and transmit the state information of the battery 330 to the processor 310. The processor 310 may provide the state information of the battery 330 to the user through an output module described below.

The IMU 360 may obtain or measure acceleration information (or posture information) of the user. For example, the IMU 360 may measure or obtain 3-axis (e.g., X-axis, Y-axis, and Z-axis) accelerations and rotation angles (e.g., roll, pitch, and yaw) according to a walking motion of the user. The IMU 360 may transmit the obtained acceleration information (e.g., the measured 3-axis accelerations and rotation angles) to the processor 310.

The processor 310 may control the wearable devices 300 and 300-1 overall.

The processor 310 may, for example, control the components (e.g., the motor driver circuits 370 and 370-1, etc.) in the wearable devices 300 and 300-1 by executing software (e.g., a program or instructions) stored in the memory 350, and perform various data processing or computation. As at least a portion of the data processing or computation, the processor 310 may store data received from other components (e.g., the IMU 360, the angle sensors 320 and 320-1, etc.) in the memory 350, and process the instructions or data stored in the memory 350.

The processor 310 may determine control information for generating a torque of each of the motors 380 and 380-1 and may control the motor driver circuits 370 and 370-1 based on the determined control information. For example, the processor 310 may determine a state factor y(t) indicating a state of a motion of the user according to an equation y(t)=sin (q_r(t))−sin(q_l(t)). q_l(t) may denote the first angle information, and q_r(t) may denote the second angle information. The processor 310 may determine control information τ(t) according to an equation τ(t)=κy(t−Δt). A gain κ may be a parameter indicating the magnitude and direction of an output torque. As the value of the gain κ increases, a greater torque may be output. If the gain κ is negative, a torque acting as a resistance force may be output to the user, and if the gain κ is positive, a torque acting as an assistance force may be output to the user. A delay Δt may be a parameter associated with a torque output timing. The value of the gain κ and the value of the delay Δt may be preset, and may be adjustable by the user, the wearable device 300, or the user terminal 110 paired with the wearable device 300. The processor 310 may determine control information τᵣ(t) for generating a torque from the motor 380-1 according to an equation τᵣ(t)=τ(t) and determine control information τₗ(t) for generating a torque from the motor 380 according to τₗ(t)=−τ(t).

The motor driver circuits 370 and 370-1 may control the motors 380 and 380-1, respectively, based on the control information received from the processor 310, and the motors 380 and 380-1 may each generate a torque by this control.

The communication module 390 may support establishing a direct (e.g., wired) communication channel or wireless communication channel between the wearable devices 300 and 300-1 and an external electronic device and performing communication through the established communication channel. The communication module may include one or more communication processors configured to support direct (or wired) communication or wireless communication. According to an embodiment, the communication module may include a wireless communication module (e.g., a cellular communication module, a short-range wireless communication module, or a global navigation satellite system (GNSS) communication module) or a wired communication module (e.g., a local area network (LAN) communication module or a power line communication (PLC) module). A corresponding one of these communication modules may communicate with the external electronic device via a first network (e.g., a short-range communication network such as Bluetooth™, Wi-Fi direct, or infrared data association (IrDA)) or a second network (e.g., a legacy cellular network, a 5G network, a next-generation communication network, the Internet, or a computer network). These various types of communication modules may be implemented as a single component (e.g., a single chip), or may be implemented as multiple components (e.g., multiple chips) separate from each other.

In an embodiment, the wearable devices 300 and 300-1 may include a display module. The display module may include, for example, a display and/or a lighting unit (e.g., the lighting unit 60 of FIG. 2A). The processor 310 may control the display module so that the display module may provide visual feedback to the user.

In an embodiment, the wearable devices 300 and 300-1 may include a sound output module. The sound output module may include, for example, a speaker. The processor 310 may control the sound output module so that the sound output module may provide auditory feedback to the user.

In an embodiment, the wearable devices 300 and 300-1 may include a vibration output module. The vibration output module may include, for example, a vibration motor. The processor 310 may control the vibration output module so that the vibration output module may provide tactile feedback (or haptic feedback) to the user.

In an embodiment, at least one of the processor 310, the battery 330, the PMIC 340, the memory 350, the IMU 360, the communication module 390, the display module, the sound output module, or the vibration output module, or a combination thereof may be positioned in the base body 10 of FIGS. 2A and 2B.

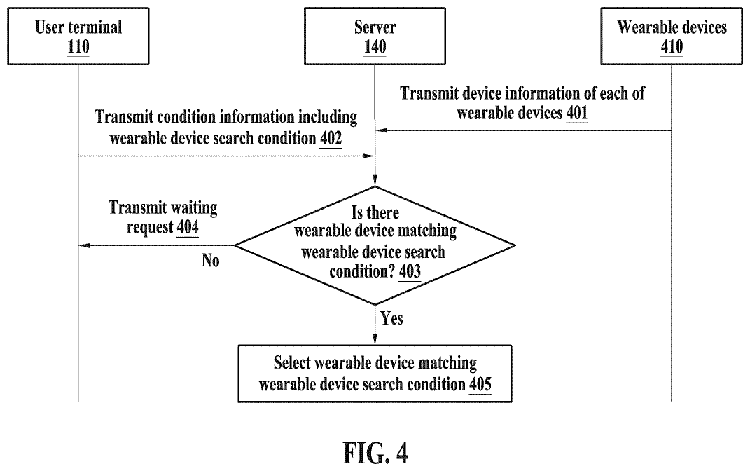

FIG. 4 is a flowchart illustrating an example of operations of a user terminal, a server, and wearable devices, according to an embodiment.

Referring to FIG. 4, in operation 401, each of wearable devices 410 may transmit device information of each of the wearable devices 410. Each of the wearable devices 410 may correspond to a wearable device 120, 200, 300, 300-1.

The device information may include, for example, a charge state and/or the size of each of the wearable devices 410. The size thereof may include, for example, size S (small), size M (medium), size L (large), size XL (extra large), and the like.

At least some or all of the wearable devices 410 may be positioned at the same location. For example, at least some or all of the wearable devices 410 may be positioned in the same gym (fitness center).

In operation 402, the user terminal 110 may transmit, to the server 140, condition information including a wearable device search condition. The wearable device search condition may be a condition for searching for a wearable device that best suits a user. The wearable device search condition may include, for example, at least one or a combination of an exercise type, an exercise time, or a size input by the user.

For example, the user terminal 110 may execute an application. The user may input, to the user terminal 110, the type of exercise the user desires to perform, an exercise duration, and the size of the user. The input size may be, for example, size S, size M, size L, or size XL, but embodiments are not limited thereto. The user terminal 110 may transmit, to the server 140, condition information including at least one or a combination of the input exercise type, the input exercise duration, or the input size. According to an embodiment, the condition information may include a location (or place) (e.g., a fitness center, etc.) at which the user performs an exercise while wearing a wearable device.

In operation 403, based on the condition information and the device information of each of the wearable devices 410, received from the user terminal 110, the server 140 may determine, among the wearable devices 410, whether there is a wearable device matching the wearable device search condition.

For example, based on the received condition information (e.g., exercise type, exercise duration, and size) and the device information (e.g., charge state and size) of each of the wearable devices 410, the server 140 may determine, among the wearable devices 410, whether there is a wearable device that may be used for the exercise duration or longer and has a size that matches the size of the user. Operation 403 is described below.

When there is no wearable device matching the wearable device search condition among the wearable devices 410, the server 140 may transmit a waiting request to the user terminal 110 in operation 404.

When there is a wearable device matching the wearable device search condition among the wearable devices 410, the server 140 may select the wearable device matching the wearable device search condition in operation 405. The server 140 may include the selected wearable device in a list. There may be one or more wearable devices matching the wearable device search condition.

The server 140 may transmit, to the selected wearable device, a control signal to provide the user with a notification indicating that the selected wearable device matches the wearable device search condition. In other words, the server 140 may transmit, to the selected wearable device, a control signal such that the selected wearable device may provide a notification (or feedback) to the user.

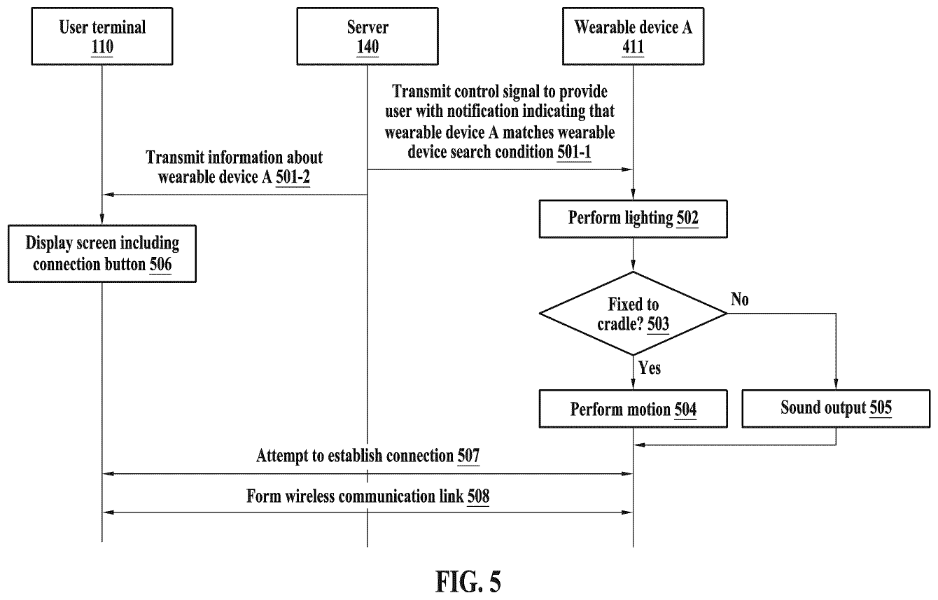

FIG. 5 is a flowchart illustrating an example of an operation of a wearable device matching a user terminal, a server, and a search condition, according to an embodiment.

In the example illustrated in FIG. 5, the server 140 may determine, among the wearable devices 410, that a wearable device A 411 matches the wearable device search condition.

In operation 501-1, the server 140 may transmit, to the wearable device A 411, a control signal to provide the user with a notification indicating that the wearable device A 411 matches the search condition.

In operation 501-2, the server 140 may transmit, to the user terminal 110, information about the wearable device A 411. The information about the wearable device A 411 may include, for example, at least one or a combination of identification information of the wearable device A 411, information about a motion being performed by the wearable device A 411, the size of the wearable device A 411, or the charge state of the wearable device A 411.

In operation 506, the user terminal 110 may display a screen, which includes a connection button, on the display of the user terminal 110. When receiving, from the server 140, the information about the wearable device A 411, the user terminal 110 may display the screen, which includes the connection button, on the display of the user terminal 110. The information about the wearable device A 411 may be displayed on the screen.

When receiving, from the server 140, the control signal to provide the user with the notification indicating that the wearable device A 411 matches the wearable device search condition, the wearable device A 411 may perform lighting in operation 502. For example, the wearable device A 411 may perform lighting by outputting light through a plurality of light sources.

In operation 503, the wearable device A 411 may determine whether the wearable device A 411 is fixed to a cradle. For example, the wearable device A 411 may determine, through a sensor, whether the wearable device A 411 is safely fixed (or fastened) to the cradle. The sensor may include, for example, a proximity sensor, but embodiments are not limited thereto.

When determining that the wearable device A 411 is fixed to the cradle, the wearable device A 411 may perform a motion in operation 504. For example, the wearable device A 411 may determine a motion pattern corresponding to an instruction (e.g., providing a notification) of the received control signal. However, embodiments are not limited thereto, and the motion pattern may be designated (or determined) in advance. The wearable device A 411 may drive an actuator (e.g., at least one of the first actuator and the second actuator described with reference to FIG. 2A) to perform a motion according to the determined motion pattern (or a designated motion pattern).

When the wearable device A 411 is fixed to the cradle, the wearable device A 411 may notify the user that the wearable device A 411 matches the wearable device search condition of the user by performing lighting and a motion. In other words, the wearable device A 411 may notify the user that the wearable device the user is looking for is the wearable device A 411. The user may clearly recognize that the wearable device A 411 among the wearable devices 410 matches the wearable device search condition of the user.

When determining that the wearable device A 411 is not fixed to the cradle, the wearable device A 411 may perform sound output in operation 505. The wearable device A 411 may output sound through a speaker. When the wearable device A 411 is not fixed to the cradle, the wearable device A 411 may notify the user that the wearable device A 411 matches the wearable device search condition of the user by performing lighting and sound output. The user may clearly recognize that the wearable device A 411 matches the wearable device search condition of the user.

The user terminal 110 may attempt to connect to the wearable device A 411 in operation 507 when there is a user input for the connection button on the screen.

In operation 508, the user terminal 110 may form a wireless communication link (e.g., a Bluetooth link) with the wearable device A 411.

According to an embodiment, the server 140 may select a plurality of wearable devices that match the wearable device search condition among the wearable devices 410. For example, the server 140 may determine that the wearable device A 411, a wearable device B, and a wearable device C match the wearable device search condition. The server 140 may transmit, to each of the wearable device A 411, the wearable device B, and the wearable device C, a control signal to provide the user with a notification indicating that each of the wearable device A 411, the wearable device B, and the wearable device C matches the wearable device search condition. Each of the wearable device A 411, the wearable device B, and the wearable device C may provide a notification to the user. For example, the wearable device A 411, the wearable device B, and the wearable device C may each perform operations 502, 503, and 504, or operations 502, 503, and 505.

The server 140 may transmit, to the user terminal 110, information about each of the wearable device A 411, the wearable device B, and the wearable device C. The information about each of the wearable device A 411, the wearable device B, and the wearable device C may include, for example, identification information about each of the wearable device A 411, the wearable device B, and the wearable device C, information about a motion performed by each of the wearable device A 411, the wearable device B, and the wearable device C, and the like, but embodiments are not limited thereto.

The user terminal 110 may display, on the display of the user terminal 110, the screen including the connection button and the information about each of the wearable device A 411, the wearable device B, and the wearable device C. The user may select one of the wearable device A 411, the wearable device B, and the wearable device C on the screen of the user terminal 110 and apply an input to the connection button. In this case, the user terminal 110 may attempt to establish a connection with the wearable device selected by the user and form a wireless communication link with the wearable device selected by the user. As described below, among the wearable device A 411, the wearable device B, and the wearable device C, wearable devices that are not connected to the user terminal 110 may stop providing a notification under the control by the server 140.

According to an embodiment, when a wireless communication link is formed between the user terminal 110 and the wearable device A 411 or when the user wears the wearable device A 411, the wearable device A 411 may stop providing a notification (e.g., performing a motion+performing lighting or outputting sound+performing lighting).

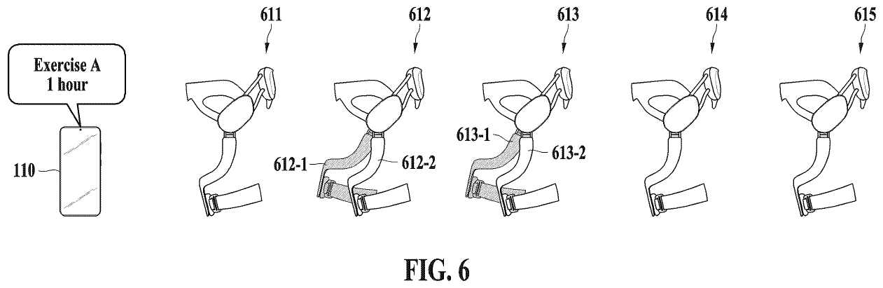

FIG. 6 is a diagram illustrating an example of a motion of a wearable device matching a search condition related to an exercise, according to an embodiment.

Referring to FIG. 6, the user terminal 110 may receive, from the user, an exercise type (e.g., exercise A) and an exercise duration (e.g., 1 hour). The user terminal 110 may transmit, to the server 140, the wearable device search condition (e.g., exercise A and the exercise duration (1 hour)). The user terminal 110 may transmit, to the server 140, a request for searching for a usable wearable device for the user to perform the exercise A for 1 hour among wearable devices 611 to 615 at a place (e.g., a fitness center) in which the user performs the exercise A.

The server 140 may receive, from the user terminal 110, the wearable device search condition (e.g., exercise A and 1 hour).

The server 140 may receive device information (e.g., a charge state) from each of the wearable devices 611 to 615. Each of the wearable devices 611 to 615 may correspond to the wearable device 120, 200, 300, 300-1.

Based on the charge state, the exercise type (e.g., exercise A), and the exercise duration (e.g., 1 hour) of each of the wearable devices 611 to 615, the server 140 may determine, among the wearable devices 611 to 615, whether there is a wearable device that may be used for the exercise duration (e.g., 1 hour) or longer. For example, using wearable device power consumption set for an exercise type (e.g., exercise A) and the charge state of each of the wearable devices 611 to 615, the server 140 may calculate an estimated usable time of each of the wearable devices 611 to 615 and determine whether there is a wearable device with an estimated usable time greater than or equal to the exercise duration.

Table 1 below shows examples of wearable device power consumption for different exercise types.

TABLE 1

| | Wearable device power consumption |
|---|---|
| | per unit time (e.g., power consumption |
| | at metabolic equivalent |
| Exercise type | (MET)/repetition (rep) (peak intensity) |
| Exercise A | 100 |
| Exercise B | 50 |
| . . . | . . . |

As shown in Table 1 above, the server 140 may identify that the wearable device power consumption set for the exercise A is 100. When the charge state of the wearable device 611 is, for example, 50%, the server 140 may calculate the estimated usable time of the wearable device 611 as 0.5 hours (=50/100). When the charge state of the wearable device 612 and the charge state of the wearable device 613 are, for example, 100%, the server 140 may calculate the estimated usable time of each of the wearable device 612 and the wearable device 613 as 1 hour (=100/100). When the charge state of the wearable device 614 and the charge state of the wearable device 615 are 80%, the server 140 may calculate the estimated usable time of each of the wearable device 614 and the wearable device 615 as 0.8 hours (=80/100).

The server 140 may determine, among the wearable devices 611 to 615, that the wearable device 612 and the wearable device 613 have an estimated usable time greater than or equal to the exercise duration (e.g., 1 hour). The server 140 may determine that the wearable device 612 and the wearable device 613 match the wearable device search condition (e.g., exercise A and 1 hour).

The server 140 may select the wearable device 612 and the wearable device 613 and include the wearable device 612 and the wearable device 613 in a list.

The server 140 may transmit, to each of the wearable device 612 and the wearable device 613, a control signal to provide the user with a notification indicating that the wearable device 612 and the wearable device 613 match the wearable device search condition (e.g., exercise A and 1 hour).

The server 140 may transmit, to the user terminal 110, information (e.g., identification information of each of the wearable device 612 and the wearable device 613, information about a motion performed by each of the wearable device 612 and the wearable device 613, etc.) about each of the wearable device 612 and the wearable device 613.

The wearable device 612 and the wearable device 613 may perform the operations of the wearable device A 411 described with reference to FIG. 5.

When receiving a control signal from the server 140, the wearable device 612 may perform lighting through light sources. When determining that the wearable device 612 is stably fixed to a cradle, the wearable device 612 may perform a motion. For example, as in the example illustrated in FIG. 6, the wearable device 612 may cause a second connection frame 612-1 (e.g., the second connection frame 41a of FIG. 2A) to move (or rotate) by generating a torque through a second driving module (e.g., the second driving module 30b of FIG. 2A). According to an embodiment, the wearable device 612 may cause the second connection frame 612-1 to continuously move, or the second connection frame 612-1 may rotate by a predetermined angle and stay in the rotation state without moving anymore.

The motion of the wearable device 612 is not limited to the above examples. The wearable device 612 may cause a first connection frame 612-2 (e.g., the first connection frame 40a of FIG. 2A) to move (or rotate) by generating a torque through a first driving module (e.g., the first driving module 30a of FIG. 2A) or cause the first connection frame 612-1 and the second connection frame 612-2 to move (or rotate) by generating a torque through the first driving module and the second driving module.

When receiving a control signal from the server 140, the wearable device 613 may perform lighting through light sources. When determining that the wearable device 613 is stably fixed to the cradle, the wearable device 613 may perform a motion. For example, as in the example illustrated in FIG. 6, the wearable device 613 may cause a second connection frame 613-1 (e.g., the second connection frame 41a of FIG. 2A) to move (or rotate) by generating a torque through the second driving module (e.g., the second driving module 30b of FIG. 2A). However, embodiments are not limited thereto, and the wearable device 613 may cause a first connection frame 613-2 (e.g., the first connection frame 40a of FIG. 2A) to move by generating a torque through the first driving module (e.g., the first driving module 30a of FIG. 2A) or cause the first connection frame 613-1 and the second connection frame 613-2 to move by generating a torque through the first driving module and the second driving module.

The wearable device 613 may perform a motion that is the same as or different from that of the wearable device 612.

Since the other wearable devices 611, 614, and 615 do not perform motions, the user may easily recognize that the wearable devices 612 and 613 match the wearable device search condition of the user.

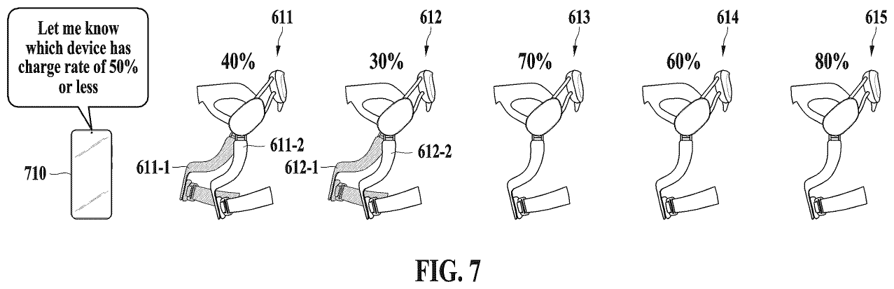

FIG. 7 is a diagram illustrating an example of a motion of a wearable device matching a search condition related to a charge rate, according to an embodiment.

Referring to FIG. 7, an administrator terminal 710 may receive a charge rate (e.g., 50%) from an administrator. The administrator may be a person who manages the wearable devices 611 to 615. The administrator terminal 710 may transmit, to the server 140, a wearable device search condition (e.g., a charge rate of 50% or less). The administrator terminal 710 may transmit, to the server 140, a request for searching for a wearable device with a charge rate of 50% or less among the wearable devices 611 to 615.

The server 140 may receive, from the administrator terminal 710, the wearable device search condition (e.g., a charge rate of 50% or less).

The server 140 may receive device information (e.g., a charge state) from each of the wearable devices 611 to 615.

As in the example illustrated in FIG. 7, the charge states of the wearable devices 611 to 615 may be 40%, 30%, 70%, 60%, and 80%, respectively. The server 140 may determine that the wearable devices 611 and 612 match the wearable device search condition (e.g., a charge rate of 50% or less).

The server 140 may select the wearable devices 611 and 612 and include the wearable devices 611 and 612 in a list.

The server 140 may transmit, to each of the wearable devices 611 and 612, a control signal to provide the administrator with a notification indicating that the wearable devices 611 and 612 match the wearable device search condition (e.g., a charge rate of 50% or less).

Each of the wearable devices 611 and 612 of FIG. 7 may perform the operations of the wearable device A 411 described with reference to FIG. 5.

When receiving the control signal from the server 140, the wearable device 611 may perform lighting through light sources. When determining that the wearable device 611 is stably fixed to the cradle, the wearable device 611 may perform a motion. For example, as in the example illustrated in FIG. 7, the wearable device 611 may cause a second connection frame 611-1 (e.g., the second connection frame 41a of FIG. 2A) to move (or rotate) by generating a torque through a second driving module (e.g., the second driving module 30b of FIG. 2A). According to an embodiment, the wearable device 612 may cause a second connection frame 612-1 to continuously move, or the second connection frame 612-1 may rotate by a predetermined angle and stay in the rotation state without moving anymore.

The motion of the wearable device 611 of FIG. 7 is not limited to the above examples. The wearable device 611 may cause a first connection frame 611-2 (e.g., the first connection frame 40a of FIG. 2A) to move (or rotate) by generating a torque through a first driving module (e.g., the first driving module 30a of FIG. 2A) or cause the first connection frame 611-1 and the second connection frame 611-2 to move (or rotate) by generating a torque through the first driving module and the second driving module.

The wearable device 611 of FIG. 7 may perform a motion different from the motion of the wearable device 612 of FIG. 6. However, embodiments are not limited thereto, and the wearable device 611 of FIG. 7 may perform the same motion as the wearable device 612 of FIG. 6.

The wearable device 612 of FIG. 7 may perform a motion that is the same as or different from that of the wearable device 611.

In the example illustrated in FIG. 7, since the other wearable devices 613, 614, and 615 do not perform motions and lighting, the administrator may easily recognize that the wearable devices 611 and 612 match the wearable device search condition (e.g., a charge rate of 50% or less) of the administrator.

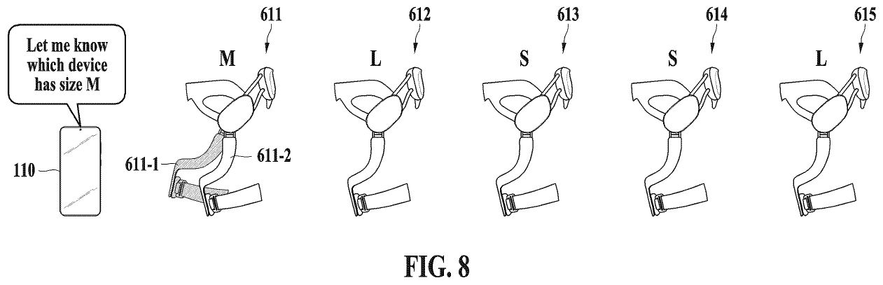

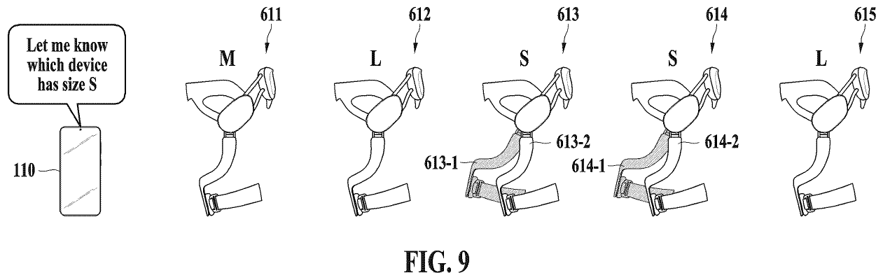

FIGS. 8 and 9 are diagrams illustrating a motion of a wearable device matching a search condition related to a size, according to an embodiment.

Referring to FIG. 8, the user terminal 110 may receive a size (e.g., size M) from a user. The user terminal 110 may transmit a wearable device search condition (e.g., size M) to the server 140. The user terminal 110 may transmit, to the server 140, a request for searching for a wearable device having size M among the wearable devices 611 to 615 at a place (e.g., a fitness center) in which the user performs an exercise.

The server 140 may receive the wearable device search condition (e.g., size M) from the user terminal 110.

The server 140 may receive device information (e.g., size) from each of the wearable devices 611 to 615. According to an embodiment, the size of each of the wearable devices 611 to 615 may be recorded in a database.

In the example illustrated in FIG. 8, the sizes of the wearable devices 611 to 615 may be size M, size L, size S, size S, and size L, respectively.

The server 140 may determine that the wearable device 611 among the wearable devices 611 to 615 matches the wearable device search condition (e.g., size M).

The server 140 may select the wearable device 611 and include the wearable device 611 in a list.

The server 140 may transmit, to the wearable device 611, a control signal to provide a user with a notification indicating that the wearable device 611 matches the wearable device search condition (e.g., size M).

The server 140 may transmit, to the user terminal 110, information (e.g., identification information of the wearable device 611, information about a motion performed by the wearable device 611, etc.) about the wearable device 611.

The wearable device 611 of FIG. 8 may perform the operations of the wearable device A 411 described with reference to FIG. 5.

When receiving the control signal from the server 140, the wearable device 611 may perform lighting through light sources. When determining that the wearable device 611 is stably fixed to the cradle, the wearable device 611 may perform a motion. For example, as in the example illustrated in FIG. 8, the wearable device 611 may cause the second connection frame 611-1 (e.g., the second connection frame 41a of FIG. 2A) to move (or rotate) by generating a torque through the second driving module (e.g., the second driving module 30b of FIG. 2A). According to an embodiment, the wearable device 611 may cause the second connection frame 611-1 to continuously move, or the second connection frame 611-1 may rotate by a predetermined angle and stay in the rotation state without moving anymore.

The motion of the wearable device 611 is not limited to the above examples. The wearable device 611 may cause the first connection frame 611-2 (e.g., the first connection frame 40a of FIG. 2A) to move (or rotate) by generating a torque through the first driving module (e.g., the first driving module 30a of FIG. 2A) or cause the first connection frame 611-1 and the second connection frame 611-2 to move (or rotate) by generating a torque through the first driving module and the second driving module.

The wearable device 611 of FIG. 8 may perform a motion different from the motion of the wearable device 612 of FIG. 6 and the motion of the wearable device 611 of FIG. 7. However, embodiments are not limited thereto, and the motion of the wearable device 611 of FIG. 8, the motion of the wearable device 612 of FIG. 6, and the motion of the wearable device 611 of FIG. 7 may be the same.

In the example illustrated in FIG. 8, since the other wearable devices 612, 613, 614, and 615 do not perform motions and lighting, the user may easily recognize that the wearable device 611 matches the wearable device search condition (e.g., size M) of the user.

According to an embodiment, the user terminal 110 may receive, from the user, an exercise type (e.g., exercise A), an exercise duration (e.g., 1 hour), and a size (e.g., size M). The user terminal 110 may transmit, to the server 140, the wearable device search condition (e.g., exercise A, exercise duration (1 hour), and size M). The user terminal 110 may transmit, to the server 140, a request for searching for a wearable device that has a size M and may be used by the user performing the exercise A for 1 hour among the wearable devices 611 to 615 at a place (e.g., a fitness center) in which the user performs the exercise A.

The server 140 may receive the wearable device search condition (e.g., exercise A, exercise duration (1 hour), and size M) from the user terminal 110.

As described with reference to FIG. 6, the server 140 may determine, based on the charge state, the exercise type (e.g., exercise A), and the exercise duration (e.g., 1 hour) of each of the wearable devices 611 to 615, whether there is a wearable device that may be used for the exercise duration or longer among the wearable devices 611 to 615.

The server 140 may determine, among the wearable devices 611 to 615, whether there is a wearable device that has size M as per the user's search request.

The server 140 may determine, among the wearable devices 611 to 615, that the wearable device 611 matches the wearable device search condition (e.g., exercise A, exercise duration (1 hour), and size M).

The server 140 may select the wearable device 611 and include the wearable device 611 in a list.

The server 140 may transmit, to the wearable device 611, a control signal to provide the user with a notification indicating that the wearable device 611 matches the wearable device search condition (e.g., exercise A, exercise duration (1 hour), and size M).

The server 140 may transmit, to the user terminal 110, information (e.g., identification information of the wearable device 611, information about a motion performed by the wearable device 611, etc.) about the wearable device 611.

When receiving the control signal from the server 140, the wearable device 611 may perform lighting through light sources. When determining that the wearable device 611 is stably fixed to the cradle, the wearable device 611 may perform a motion.

Since the other wearable devices 612, 613, 614, and 615 do not perform motions and lighting, the user may easily recognize that the wearable device 611 matches the wearable device search condition (e.g., exercise A, exercise duration (1 hour), and size M) of the user.

Referring to FIG. 9, the administrator terminal 710 may receive a size (e.g., size S) from an administrator. The administrator terminal 710 may transmit a wearable device search condition (e.g., size S) to the server 140. The administrator terminal 710 may transmit, to the server 140, a request for searching for a wearable device having size S among the wearable devices 611 to 615.

The server 140 may receive the wearable device search condition (e.g., size S) from the administrator terminal 710.

In the example illustrated in FIG. 9, the sizes of the wearable devices 611 to 615 may be size M, size L, size S, size S, and size L, respectively.

The server 140 may determine, among the wearable devices 611 to 615, that the wearable devices 614 and 614 match the wearable device search condition (e.g., size S) of the administrator.

The server 140 may select the wearable devices 613 and 614 and include the wearable devices 613 and 614 in a list.

The server 140 may transmit, to the wearable devices 613 and 614, a control signal to provide the administrator with a notification indicating that the wearable devices 613 and 614 match the wearable device search condition (e.g., size S).

Each of the wearable devices 613 and 614 of FIG. 9 may perform the operations of the wearable device A 411 described with reference to FIG. 5.

When receiving a control signal from the server 140, the wearable device 613 may perform lighting through light sources. When determining that the wearable device 613 is stably fixed to the cradle, the wearable device 613 may perform a motion. For example, as in the example illustrated in FIG. 9, the wearable device 613 may cause the second connection frame 613-1 (e.g., the second connection frame 41a of FIG. 2A) to move (or rotate) by generating a torque through a second driving module (e.g., the second driving module 30b of FIG. 2A). According to an embodiment, the wearable device 613 may cause the second connection frame 613-1 to continuously move, or the second connection frame 613-1 may rotate by a predetermined angle and stay in the rotation state without moving anymore.

The motion of the wearable device 613 is not limited to the above examples. The wearable device 613 may cause the first connection frame 613-2 (e.g., the first connection frame 40a of FIG. 2A) to move (or rotate) by generating a torque through a first driving module (e.g., the first driving module 30a of FIG. 2A) or may cause the first connection frame 613-1 and the second connection frame 613-2 to move (rotate) by generating a torque through the first and second driving modules.

The wearable device 613 of FIG. 9 may perform a motion different from each of the motion of the wearable device 612 of FIG. 6 and the motion of the wearable device 611 of FIG. 7. However, embodiments are not limited thereto, and the motion of the wearable device 613 of FIG. 9, the motion of the wearable device 612 of FIG. 6, and the motion of the wearable device 611 of FIG. 7 may be the same.

When receiving a control signal from the server 140, the wearable device 614 may perform lighting. The wearable device 614 may determine whether the wearable device 614 is fixed to a cradle. When determining that the wearable device 614 is fixed to the cradle, the wearable device 614 may cause the second connection frame 614-1 (e.g., the second connection frame of FIG. 2A) to move (or rotate) by generating a torque through a second driving module (e.g., the second driving module 30b of FIG. 2A). However, embodiments are not limited thereto, and the wearable device 614 may cause a first connection frame 614-2 (e.g., the first connection frame 40a of FIG. 2A) to move (or rotate) by generating a torque through the first driving module (e.g., the first driving module 30a of FIG. 2A) or cause the first connection frame 614-1 and the second connection frame 614-2 to move (rotate) by generating a torque through the first driving module and the second driving module.

In the example illustrated in FIG. 9, since the other wearable devices 611, 612, and 615 do not perform motions and lighting, the administrator may easily recognize that the wearable devices 613 and 614 match the wearable device search condition (e.g., size S) of the administrator.

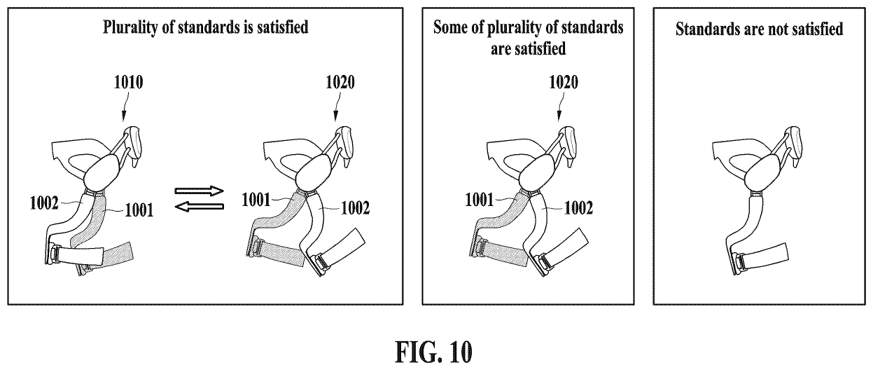

FIG. 10 is a diagram illustrating an example of a wearable device performing different motions depending on the number of satisfied standards, according to an embodiment.

Referring to FIG. 10, a motion (or motion pattern) of a wearable device (e.g., the wearable device 120, 200, 300, 300-1) may vary depending on the number of satisfied standards.

In an embodiment, the server 140 may receive, from the user terminal 110, condition information including a wearable device search condition (e.g., an exercise type, an exercise duration, and a size input by a user). The server 140 may receive device information (e.g., a charge state and/or a size) from each of the wearable devices 410 (e.g., the wearable devices 611 to 615). According to an embodiment, the size of each of the wearable devices 410 may be recorded in a database. The server 140 may identify the size of each of the wearable devices 410 through a database without receiving the size from each of the wearable devices 410.

Based on the received condition information (e.g., an input exercise type, an input exercise duration, and an input size), the charge state of each of the wearable devices 410, and the size of each of the wearable devices 410, the server 140 may select a first wearable device satisfying a plurality of standards and a second wearable device satisfying some of the plurality of standards. The plurality of standards may include, for example, a first standard that is satisfied when a device size and a size input by the user match each other and a second standard that is satisfied when wearable device power consumption set for an exercise type input by the user and an estimated usable time calculated using the charge state of each of the wearable devices 410 are greater than an exercise duration input by the user.

For example, in the example illustrated in FIG. 8, the server 140 may receive, from the user terminal 110, a wearable device search condition (e.g., exercise A, exercise duration (1 hour), and size M). The server 140 may check whether the size (e.g., size M) of the wearable device 611 among the wearable devices 611 to 615 is the same as the size (e.g., size M) input by the user. The server 140 may determine that the wearable device 611 satisfies the first standard.

Using the wearable device power consumption (e.g., 100 in Table 1 above) set for the exercise type (e.g., exercise A) and the charge state of each of the wearable devices 611 to 615, the server 140 may calculate the estimated usable time of each of the wearable devices 611 to 615. The server 140 may determine that the estimated usable time of each of the wearable devices 611 and 612 among the wearable devices 611 to 615 is greater than or equal to the exercise duration (e.g., 1 hour) input by the user. The server 140 may determine that each of the wearable devices 611 and 612 satisfies the second standard.

The server 140 may determine that the wearable device 611 satisfies all of the plurality of standards (e.g., the first standard and the second standard) and may determine that the wearable device 612 satisfies some (e.g., the second standard) of the plurality of standards. The server 140 may determine that the remaining wearable devices 613, 614, and 615 do not satisfy all of the plurality of standards.

The server 140 may transmit, to the wearable device 611, a first control signal to notify the user that the wearable devices 611 satisfies the plurality of standards. The server 140 may transmit, to the wearable device 612, a second control signal to notify the user that the wearable device 612 satisfies some of the plurality of standards.

When receiving the first control signal from the server 140, the wearable device 611 may perform a notification corresponding to the first control signal. For example, the wearable device 611 may perform first lighting by outputting light of a first color through light sources. When determining that the wearable device 611 is stably fixed to a cradle, the wearable device 611 may cause the first connection frame 611-1 and the second connection frame 611-2 to move by generating a torque through a driving module (e.g., the driving module 30 of FIG. 2A).

For example, as in the example illustrated in FIG. 10, the wearable device 611 may be configured such that the first connection frame 611-2 (e.g., a first connection frame 1002) and the second connection frame 611-1 (e.g., a second connection frame 1001) of the wearable device 611 are in an intersecting state 1010. The wearable device 611 that satisfies the plurality of standards may cause the second connection frame 611-1 to move (or rotate) in the backward direction of the wearable device 611 by a first angle and may cause the first connection frame 611-2 of the wearable device 611 to move (or rotate) in a forward direction by the first angle. The wearable device 611 may cause the second connection frame 611-1 to move (or rotate) in the forward direction and cause the first connection frame 611-2 in the backward direction in the intersecting state 1010. Accordingly, as in the example illustrated in FIG. 10, the first connection frame 611-2 and the second connection frame 611-1 of the wearable device 611 may be in a separated state 1020. In the case of the separated state 1020, the first connection frame 611-2 may be in a state of moving (or rotating) in the backward direction by the first angle, and the second connection frame 611-1 may be in a state of moving (or rotating) in the forward direction by the first angle. The wearable device 611 may repeat the intersecting state 1010 and the separated state 1020. Additionally, although not shown in FIG. 10, the wearable device 611 may perform first lighting.

When receiving the second control signal from the server 140, the wearable device 612 may perform a notification corresponding to the second control signal. For example, the wearable device 612 may perform second lighting by causing light of a second color to blink through light sources. The blinking cycle of the light of the second color may be, for example, 1 second, but embodiments are not limited thereto. When determining that the wearable device 612 is stably fixed to the cradle, the wearable device 612 may allow the first connection frame 612-1 and the second connection frame 612-2 to stay separated by a predetermined angle.

For example, the wearable device 612 that satisfies some of the plurality of standards may cause the second connection frame 612-2 (e.g., the second connection frame 1001) to move (or rotate) by the first angle in the forward direction and may cause the first connection frame 611-2 (e.g., the first connection frame 1002) to move (or rotate) by the first angle in the backward direction. Accordingly, the first connection frame 612-2 and the second connection frame 612-1 of the wearable device 612 may be in a separated state 1020 in which the first connection frame 612-2 and the second connection frame 612-1 are separated by a predetermined angle (e.g., twice the first angle). The wearable device 612 may maintain the separated state 1020.

Each of the wearable devices 613, 614, and 615 that does not satisfy the standards may be in an idle state 1030 in which the wearable devices 613, 614, and 615 do not perform lighting or motions.

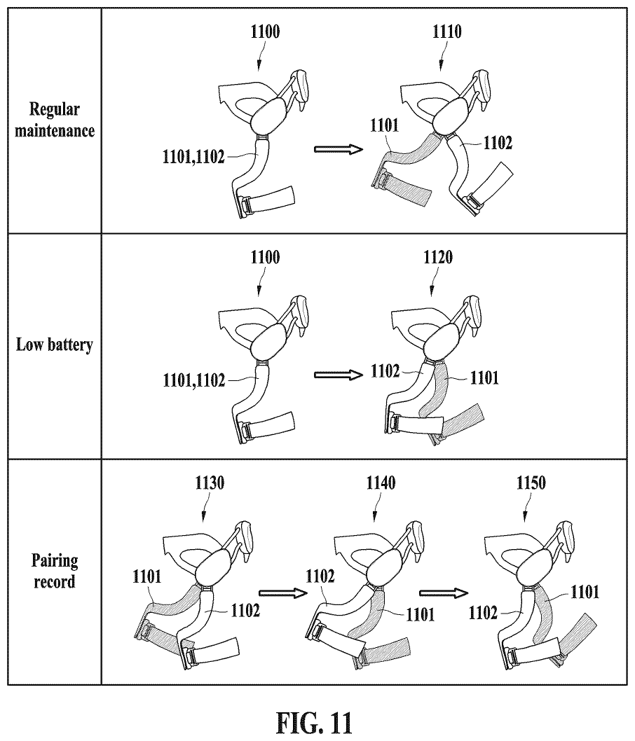

FIG. 11 is a diagram illustrating an example of performing different motions depending on the state of a wearable device, according to an embodiment.

Referring to FIG. 11, a motion (or a motion pattern) and lighting to be performed may vary depending on the state (e.g., a regular maintenance state, a charge state (or a low battery state), a state regarding a pairing record (or a wireless communication connection record)) of a wearable device (e.g., the wearable device 120, 200, 300, 300-1).

In an embodiment, the administrator terminal 710 may receive a selection input from an administrator for one of the wearable devices 410 (e.g., the wearable devices 611 to 615). The administrator terminal 710 may receive, from the administrator, a selection input regarding a state that the administrator desires to check among the states (e.g., a regular maintenance state, a charge state, and a state of a pairing record) of the wearable device (hereinafter, referred to as a “target wearable device”) selected by the administrator. The administrator terminal 710 may transmit, to the server 140, a request for checking a selected state of the target wearable device.

Based on the received check request, the server 140 may transmit, to the target wearable device, a control signal to notify the administrator of the state in which the target wearable device is selected.

In an embodiment, the server 140 may receive, from the administrator terminal 710, a request for checking a regular maintenance state of the target wearable device. The server 140 may check whether a regular maintenance is performed on the target wearable device. When the regular maintenance date of the target wearable device elapses without regular maintenance, the server 140 may transmit, to the target wearable device, a control signal to notify the administrator that the regular maintenance is not performed. When receiving the control signal from the server 140, the target wearable device may perform a motion corresponding to an unexecuted state.

For example, when receiving the control signal from the server 140, the target wearable device may perform third lighting by outputting light of a third color through light sources. The target wearable device may cause a first connection frame 1102 to move (or rotate) in a backward direction by a second angle and may cause a second connection frame 1101 to move (or rotate) in a forward direction by the second angle. The target wearable device may maintain a separated state 1110 in which the first connection frame 1102 and the second connection frame 1101 are separated by a predetermined angle (e.g., twice the second angle). When receiving, from the server 140, a control signal to notify the administrator of an unexecuted state, the target wearable device may perform a motion from the idle state 1100 to the separated state 1110 and perform the third lighting.

Depending on the embodiment, a record of whether regular maintenance is performed may be stored in the target wearable device. The server 140 may transmit, to the target wearable device, a control signal to allow the target wearable device to notify the administrator of a regular maintenance state. When receiving the control signal from the server 140, the target wearable device may check whether regular maintenance is performed. When a regular maintenance date elapses without regular maintenance, the target wearable device may perform the third lighting and a motion corresponding to a state in which the regular maintenance is not executed (e.g., the separated state 1110 in which the first connection frame 1102 and the second connection frame 1101 are kept separated by a predetermined angle).

The target wearable device may perform the third lighting and the motion corresponding to the unexecuted state for a first time (e.g., 30 seconds) when the timepoint of a request for checking the regular maintenance state is within a predetermined period (e.g., regular maintenance date+1 to regular maintenance date+7) from the timepoint when the regular maintenance date elapses (e.g., regular maintenance day+1). In other words, when there is a request for checking the regular maintenance state between, for example, the regular maintenance date+1 and the regular maintenance date+7, the target wearable device may perform the third lighting and the motion corresponding to the unexecuted state for the first time (e.g., 30 seconds). When the timepoint of the request for checking the regular maintenance state is a predetermined duration after the regular maintenance date elapses (e.g., when the regular maintenance date is overdue by more than 7 days), the target wearable device may perform the third lighting and the motion corresponding to the unexecuted state for a second time (e.g., 1 minute), which is longer than the first time. In other words, when there is a request for checking the regular maintenance state, for example, more than 7 days after the regular maintenance date, the target wearable device may perform the third lighting and the motion corresponding to the unexecuted state for the second time (e.g., 1 minute).

In an embodiment, the server 140 may receive, from the administrator terminal 710, a request for checking the charge state (or low battery state) of the target wearable device. The server 140 may transmit, to the target wearable device, a control signal to allow the target wearable device to notify the administrator of the charge state (or low battery state). When receiving the control signal from the server 140, the target wearable device may check the charge state of the battery of the target wearable device. Through this checking process, it may be determined that the target wearable device is in the low battery state. In this case, the target wearable device may perform fourth lighting and a motion corresponding to the low battery state. When determining that the target wearable device is not in the low battery state, the target wearable device may remain in the idle state.

For example, when determining that the target wearable device is in the low battery state, the target wearable device may perform the fourth lighting by causing light of the third color to blink through light sources. The target wearable device may cause the first connection frame 1102 to move (or rotate) in a forward direction by the second angle and may cause the second connection frame 1101 to move (or rotate) in a backward direction by the second angle. The target wearable device may maintain an intersecting state 1120 in which the moved first connection frame 1102 intersects with the moved second connection frame 1101. When determining that the target wearable device is in the low battery state, the target wearable device may perform the fourth lighting and a motion from the idle state 1100 to the separated state 1120.

When the charge state of the battery is less than or equal to a first level (e.g., 10%), the target wearable device may perform the fourth lighting and the motion (e.g., maintaining the intersecting state 1120 in which the first connection frame 1102 intersects with the second connection frame 1101) corresponding to the low battery state. When the charge state of the target wearable device is less than or equal to a second level (e.g., 5%), which is lower than the first level, the target wearable device may perform the fourth lighting and the motion corresponding to the low battery state for a fourth time (e.g., 1 minute).

When the charge state of the battery is greater than the first level, the target wearable device may remain in the idle state 1100.

In an embodiment, the server 140 may receive, from the administrator terminal 710, a request for checking a pairing record (or wireless communication connection record) state of the target wearable device. The server 140 may transmit, to the target wearable device, a control signal to cause the target wearable device to notify the administrator of the state of the pairing record (or wireless communication connection record). When receiving the control signal from the server 140, the target wearable device may check the pairing record of the target wearable device. When there is no pairing record, the target wearable device may perform fifth lighting and a motion corresponding to a state in which there is no pairing record.

For example, when there is no pairing record, the target wearable device may perform the fifth lighting by causing light of a first color to blink through light sources. The target wearable device may cause one connection frame (e.g., the first connection frame 1102) to move (or rotate) once in a forward direction by the second angle. The target wearable device may cause the other connection frame (e.g., the second connection frame 1101) to move (or rotate) in a backward direction twice by the second angle. States 1130, 1140, and 1150 of FIG. 11 show examples of motions corresponding to states in which there is no pairing record.

When there is no pairing record of the target wearable device during a first period (e.g., one to seven days), the target wearable device may perform the fifth lighting and the motion corresponding to the state in which there is no pairing record for a fifth time (e.g., 30 seconds). When there is no pairing record for a second period (e.g., 8 to 30 days), the target wearable device may perform the fifth lighting and the motion corresponding to the state in which there is no pairing record for a sixth time (e.g., 1 minute). When there is no pairing record for more than the second period (e.g., when there is no pairing record for 31 days or more), the target wearable device may perform the fifth lighting and the motion corresponding to the state in which there is no pairing record for a seventh time.

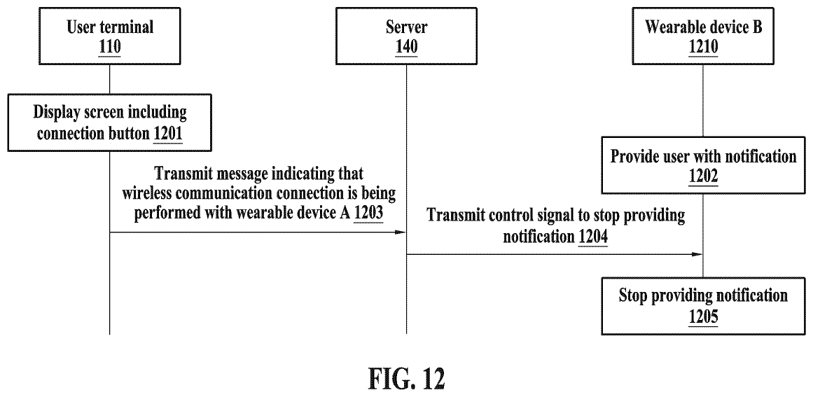

FIG. 12 is a flowchart illustrating an operation of stopping a notification from another wearable device when a user terminal is connected to a wearable device, according to an embodiment.

A wearable device B 1210 of FIG. 12 may be, together with the wearable device A 411, a wearable device matching a wearable device search condition.

Referring to FIG. 12, in operation 1201, the user terminal 110 may display a screen, which includes a connection button, on a display. For example, the screen may display information about the wearable device A 411 and the wearable device B 1210 of FIG. 5. In another example, when the server 140 recommends the wearable device A 411 to a user among the wearable device A 411 and the wearable device B 1210, information about the wearable device A 411 may be displayed on the screen.

In operation 1202, together with the wearable device A 411, the wearable device B 1210 may provide the user with a notification. When determining that the wearable device B 1210 is safely fixed to a cradle, the wearable device B 1210 may perform at least one of a motion or lighting. When determining that the wearable device B 1210 is not safely fixed to the cradle, the wearable device B 1210 may perform at least one of sound output or lighting.

When there is a user input for a connection button on the screen, the user terminal 110 may attempt to establish a connection with the wearable device A 411 of FIG. 5. For example, the user terminal 110 may receive, from the user, a selection input for the wearable device A 411 among the wearable device A 411 and the wearable device B 1210 and may attempt to establish a connection with the wearable device A 411 when there is the user input for the connection button on the screen. In another example, the user terminal 110 may display, on the screen, the wearable device A 411 recommended to the user and may attempt to establish a connection with the wearable device A 411 when there is the user input for a connection button on the screen.

In operation 1203, when there is the user input for a connection button on the screen, the user terminal 110 may transmit, to the server 140, a message indicating that a wireless communication connection is being performed with the wearable device A 411.

In operation 1204, when receiving the message from the user terminal 110, the server 140 may transmit, to the wearable device B 1210, a control signal to stop providing a notification.

In operation 1205, the wearable device B 1210 may stop providing a notification and remain in an idle state.

For example, in the example illustrated in FIG. 6, the user terminal 110 may transmit, to the server 140, a message indicating that a wireless communication connection is being performed with the wearable device 612. The server 140 may transmit, to the wearable device 613 providing a notification, the control signal to stop providing a notification. The wearable device 613 may stop providing a notification.

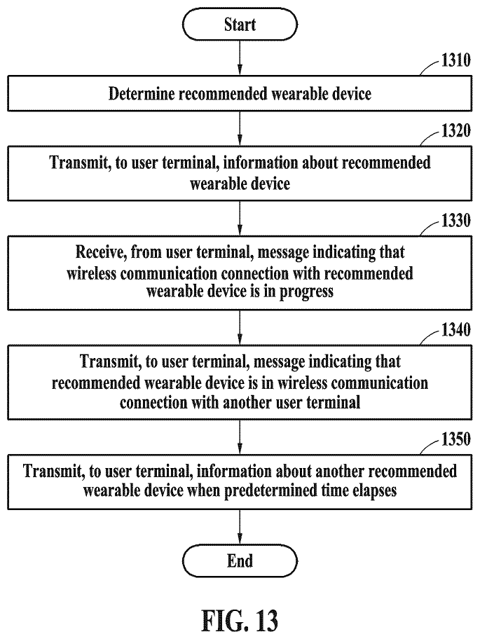

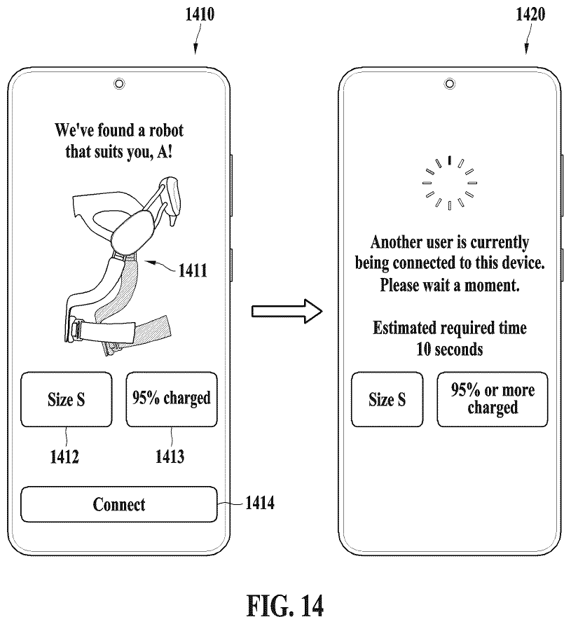

FIGS. 13 and 14 are diagrams illustrating examples of operations of a server and a user terminal when the same wearable device is recommended to a plurality of users, according to an embodiment.

Referring to FIG. 13, in operation 1310, the server 140 may determine a recommended wearable device to recommend to a user among one or more wearable devices matching a wearable device search condition. For example, the server 140 may determine the wearable device A 411 as the recommended wearable device among the wearable device A 411 and the wearable device B 1210 that matches the wearable device search condition.

In operation 1320, the server 140 may transmit, to the user terminal 110, information about the recommended wearable device (e.g., identification information of the recommended wearable device, information about a motion to be performed by the recommended wearable device, etc.). The user terminal 110 may display a screen including the information about the recommended wearable device and a connection button. An example of the screen is illustrated in FIG. 14. In the example illustrated in FIG. 14, a screen 1410 may include an image 1411 of a motion being performed by a recommended wearable device, a size 1412 of the recommended wearable device, a charge state 1413 of the recommended wearable device, and a connection button 1414. When there is a user input for the connection button 1414, the user terminal 110 may transmit a communication connection request to the recommended wearable device and transmit, to the server 140, a message indicating that a wireless communication connection with the recommended wearable device is in progress.

In operation 1330, the server 140 may receive, from the user terminal 110, a message indicating that the wireless communication connection with the recommended wearable device is in progress.

In operation 1340, the server 140 may transmit, to the user terminal 110, a message indicating that the recommended wearable device is in wireless communication connection with another user terminal. For example, the server 140 may recommend the wearable device A 411 to the user and other users. The server 140 may recommend the same wearable device to a plurality of users. When the user terminal 110 transmits, to the server 140, later than the other user terminals, a message indicating that the recommended wearable device is in wireless communication connection with the other user terminal, the server 140 may transmit, to the user terminal 110, a message indicating that the recommended wearable device is in wireless communication connection with the other user terminal.

When receiving, from the server 140, the message indicating that the recommended wearable device is in wireless communication connection with the other user terminal, the user terminal 110 may display a screen 1420 of FIG. 14 on the display.

In operation 1350, when there is another recommended wearable device, the server 140 may transmit, to the user terminal 110, information about another recommended wearable device. For example, the server 140 may determine that the wearable device B 1210 in addition to the wearable device A 411 matches the wearable device search condition of the user. In this case, the server 140 may transmit, to the user terminal 110, information about the wearable device B 1210. When an estimated required time on the screen 1420 of FIG. 14 elapses, the user terminal 110 may display, on the display, a screen including an image of a motion performed by the wearable device B 1210, the size of the wearable device B 1210, the charge state of the wearable device B 1210, and a connection button.

According to an embodiment, when there is no wearable device matching the wearable device search condition of the user except for the recommended wearable device or when there is a wearable device matching a portion of the wearable device search condition, the server 140 may transmit, to the user terminal 110, a message indicating that there is no wearable device matching the wearable device search condition or a message indicating that there is a wearable device matching a portion of the wearable device search condition. The user terminal 110 may notify the user that there is no wearable device matching the wearable device search condition or notify the user that there is a wearable device matching a portion of the wearable device search condition.

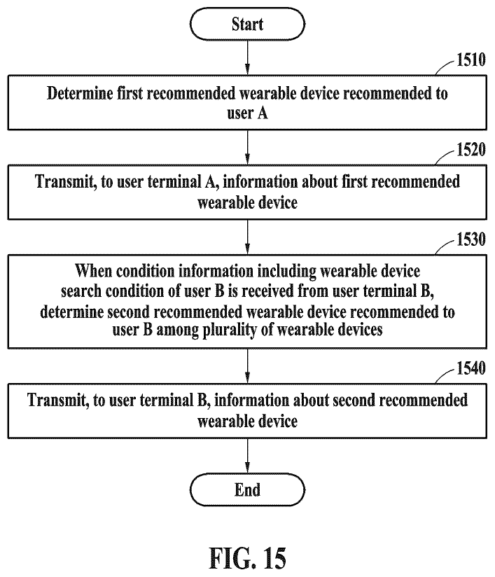

FIGS. 15, 16, and 17 are diagrams illustrating examples of operations of a user terminal, a server, and a wearable device when different wearable devices are recommended to a plurality of users, according to an embodiment.

Referring to FIG. 15, in operation 1510, the server 140 may determine a first recommended wearable device to recommend to a user (hereinafter, referred to as a “user A” in FIGS. 15, 16, and 17) from among one or more wearable devices that match a wearable device search condition. For example, the server 140 may determine the wearable device A 411 as the first recommended wearable device among the wearable device A 411 and the wearable device B 1210 that matches the wearable device search condition.

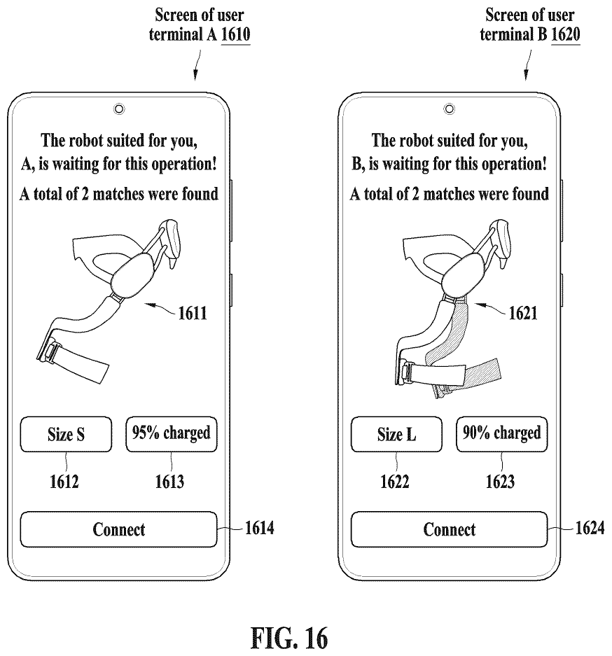

In operation 1520, the server 140 may transmit, to the user terminal 110 (hereinafter, referred to as a “user terminal A” in FIGS. 15, 16, and 17), information (e.g., identification information of the first recommended wearable device, information about a first motion performed by the first recommended wearable device, etc.) about the first recommended wearable device. The user terminal A may display a screen including the information about the first recommended wearable device and a connection button. An example of the screen of the user terminal A is illustrated in FIG. 16. In the example illustrated in FIG. 16, a screen 1610 of the user terminal A may include an image 1611 of a first motion being performed by the first recommended wearable device, a size 1612 of the first recommended wearable device, a charge state 1613 of the first recommended wearable device, and a connection button 1414. The first motion may be, for example, a motion in which first and second connection frames (e.g., the first connection frame 40a and the second connection frame 41a) of the first recommended wearable device maintain a state of rotating in a forward direction by a first angle.

The server 140 may transmit, to the first recommended wearable device, a control signal instructing to perform the first motion.

When there is an input from the user A to the connection button 1614, the user terminal A may transmit a communication connection request to the first recommended wearable device and transmit, to the server 140, a message indicating that a wireless communication connection with the first recommended wearable device is in progress.

The user A may easily recognize that the wearable device performing the first motion is the first recommended wearable device.

Referring back to FIG. 15, in operation 1530, when receiving condition information including a wearable device search condition of another user (referred to as a “user B” in FIGS. 15, 16, and 17) from another user terminal (referred to as a “user terminal B” in FIGS. 15, 16, and 17), the server 140 may determine a second recommended wearable device to recommend to the user B among the plurality of wearable devices 410. In this case, the second recommended wearable device may be different from the first recommended wearable device. When the second recommended wearable device is the same as the first recommended wearable device, the descriptions of FIGS. 13 and 14 may be applied.

In operation 1540, the server 140 may transmit, to the user terminal B, information (e.g., identification information of the second recommended wearable device, information about a second motion performed by the second recommended wearable device, etc.) about the second recommended wearable device. The second motion may be different from the first motion. The user terminal B may display a screen including the information about the second recommended wearable device and a connection button. An example of the screen of the user terminal B is illustrated in FIG. 16. In the example illustrated in FIG. 16, a screen 1620 of the user terminal B may include an image 1621 of a second motion being performed by the second recommended wearable device, a size 1622 of the second recommended wearable device, a charge state 1623 of the second recommended wearable device, and a connection button 1624. The second motion may be, for example, a motion in which the first and second connection frames (e.g., the first connection frame 40a and the second connection frame 41a) of the second recommended wearable device maintain an intersecting state.

The server 140 may transmit, to the second recommended wearable device, a control signal instructing to perform the second motion.

When there is an input from the user B to the connection button 1624, the user terminal B may transmit a communication connection request to the second recommended wearable device and transmit, to the server 140, a message indicating that a wireless communication connection with the second recommended wearable device is in progress.

The user B may easily recognize that the wearable device performing the second motion is the second recommended wearable device.

In an embodiment, a user C as well as the users A and B may desire to find a wearable device that matches a wearable device search condition of the users among the wearable devices 410. A user terminal C may receive, from the user C, a wearable device search condition of the user C. When receiving, from the user terminal C, condition information including the wearable device search condition of the user C, the server 140 may determine a third recommended wearable device to recommend to the user C among the plurality of wearable devices 410. In this case, the third recommended wearable device may be different from the first and second recommended wearable devices.

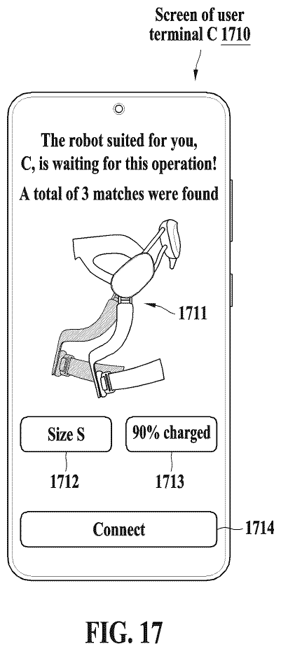

The server 140 may transmit, to the user terminal C, information (e.g., identification information of the third recommended wearable device, information about a third motion performed by the third recommended wearable device, etc.) about the third recommended wearable device. The third motion may be different from the first and second motions. The user terminal C may display a screen including the information about the third recommended wearable device and a connection button. An example of the screen of the user terminal C is illustrated in FIG. 17. In the example illustrated in FIG. 17, a screen 1710 of the user terminal C may include an image 1711 of the third motion being performed by the third recommended wearable device, a size 1712 of the third recommended wearable device, a charge state 1713 of the third recommended wearable device, and a connection button 1714. The third motion may be, for example, a motion in which the first connection frame (e.g., the first connection frame 41a) of the third recommended wearable device maintains a state of rotating by a first angle in the forward direction.

The server 140 may transmit, to the third recommended wearable device, a control signal instructing to perform the third motion.

When there is an input from the user C to the connection button 1714, the user terminal C may transmit a communication connection request to the third recommended wearable device and transmit, to the server 140, a message indicating that a wireless communication connection with the third recommended wearable device is in progress.

The user C may easily recognize that the wearable device performing the third motion is the third recommended wearable device.

According to an embodiment, when each of a plurality of users searches for a wearable device that meets a condition of each of the plurality of users among the wearable devices 410 at the same location (hereinafter, referred to as “simultaneous search by a plurality of users”), the server 140 may recommend a wearable device for each user without duplication and may cause each recommended wearable device to perform a different motion. The number of non-duplicated motions may be predetermined.

According to an embodiment, more than a given number of concurrent searches may occur. For example, the number of non-duplicated motions (e.g., the first motion, the second motion, and the third motion) may be determined as 3. In addition to the users A, B, and C, a user D may desire to find a wearable device among the wearable devices 410 that matches a wearable device search condition of the user D. A user terminal D may receive, from the user D, the wearable device search condition of the user D and transmit, to the server 140, condition information including the input wearable device search condition. When receiving the condition information from the user terminal D, the server 140 may determine a fourth recommended wearable device to recommend to the user D among the plurality of wearable devices 410. In this case, the fourth recommended wearable device may be different from the first, second, and third recommended wearable devices.

When there is a simultaneous search by a plurality of users, the server 140 may assign the non-duplicated motions (e.g., the first motion, the second motion, and the third motion) to the first, second, and third recommended wearable devices, respectively. The server 140 may not have a motion to assign to the fourth recommended wearable device. In this case, the server 140 may request the user terminal D to wait for a moment. The user terminal D may display a standby screen on a display. When the wireless communication link between the first recommended wearable device and the user terminal A is formed faster than wireless communication links between the remaining recommended wearable devices and the remaining user terminals, the server 140 may assign the first motion to the fourth recommended wearable device. The server 140 may transmit, to the user terminal D, information about the fourth recommended wearable device and transmit, to the fourth recommended wearable device, a control signal instructing to perform the first motion. The user terminal D may form a wireless communication link with the fourth recommended wearable device.

The embodiments described with reference to FIGS. 1 to 14 may apply to the embodiments of FIGS. 15, 16, and 17.

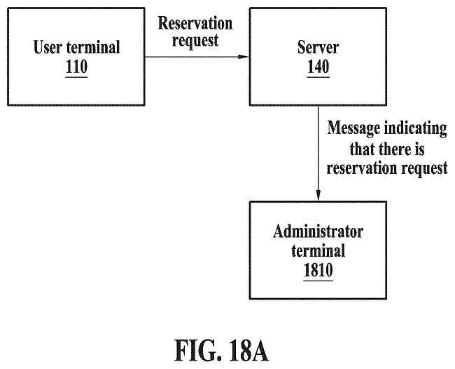

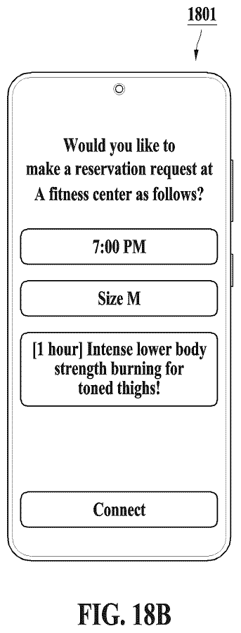

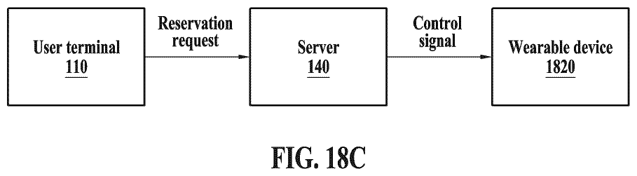

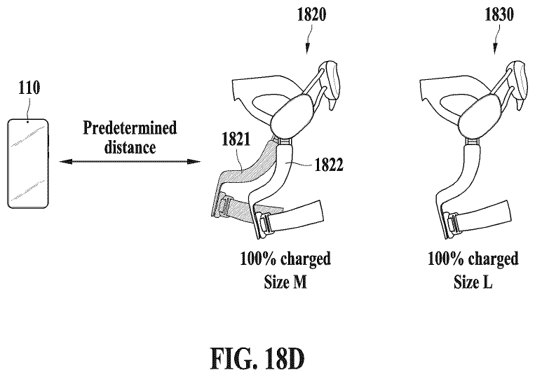

FIGS. 18A and 18B are diagrams illustrating examples of operations of a user terminal, a server, and an administrator terminal when there is a wearable device usage and exercise reservation, according to an embodiment.

Referring to FIG. 18A, the user terminal 110 may transmit, to the server 140, a reservation request for scheduling an exercise reservation. For example, the user terminal 110 may receive, from a user, at least one or a combination of a scheduled exercise time indicating when the user is expected to exercise, an exercise location (e.g., a fitness center) at which the user is expected to exercise, exercise information (e.g., an exercise duration indicating how long a user is expected to exercise and an exercise type) about the exercise to be performed, or user information (e.g., size, etc.). The user terminal 110 may generate a reservation request including information (e.g., at least one or a combination of the scheduled exercise time, the exercise location, the exercise information, or the user information) input by the user. The user terminal 110 may display a screen corresponding to the generated reservation request on a display.

An example of a screen corresponding to a reservation request is illustrated in FIG. 18B. In the example illustrated in FIG. 18B, a screen 1801 may include a scheduled exercise time (e.g., 7:00 PM), an exercise location (e.g., fitness center A), an exercise duration (e.g., 1 hour), an exercise type (e.g., lower body strength training), and a size (e.g., size M) of the user. When there is a user input to a reservation request button on the screen 1801, the user terminal 110 may transmit, to the server 140, a reservation request including the exercise start time (e.g., 7:00 PM), the exercise duration (e.g., 1 hour), the exercise type (e.g., lower body strength training), and the size (e.g., size M).

Referring back to FIG. 18A, when receiving a reservation request from the user terminal 110, the server 140 may transmit, to an administrator terminal 1810 (e.g., the administrator terminal 710), a message indicating that there is a reservation request. The administrator terminal 1810 may notify an administrator that a reservation request is made by the user. The administrator may find a wearable device suitable for the user among the wearable devices 410 (e.g., the wearable devices 611 to 615) by considering the user information (e.g., size).

FIGS. 18C and 18D are diagrams illustrating examples of operations of a user terminal, a server, and a wearable device when there is a wearable device usage and exercise reservation, according to an embodiment.

Referring to FIG. 18C, the user terminal 110 may transmit, to the server 140, a reservation request for scheduling an exercise reservation. For example, the user terminal 110 may receive, from the user, at least one or a combination of the scheduled exercise time, the exercise location, the exercise information (e.g., the exercise time, the exercise type, etc.), or the user information (e.g., size, etc.). The user terminal 110 may generate a reservation request including information (e.g., at least one or a combination of the scheduled exercise time, the exercise location, the exercise information, or the user information) input by the user. The user terminal 110 may display, on the display, a screen (e.g., a screen 1801) corresponding to the generated reservation request.

When receiving the reservation request from the user terminal 110, the server 140 may register the exercise reservation of the user. When the scheduled exercise time approaches, the server 140 may determine a wearable device suitable for the user among the wearable devices 410 (e.g., the wearable devices 611 to 615) within the exercise location included in the reservation request. The server 140 may determine a wearable device that satisfies a condition (e.g., size) of the user and a charge state among the wearable devices 410. The server 140 may determine whether any of the wearable devices 410 may be used for the exercise duration or longer based on the charge state, exercise type, and exercise duration of each of the wearable devices 410. For example, using the wearable device power consumption set for the exercise type and the charge state of each of the wearable devices 410, the server 140 may calculate the estimated usable time of each of the wearable devices 410 and determine whether there is a wearable device with an estimated usable time greater than or equal to the exercise duration. The server 140 may determine whether there is a wearable device with a size that matches the size of the user. The server 140 may determine, as a wearable device suitable for the user, a wearable device 1820 that has an estimated usable time greater than or equal to the exercise duration and a size that matches the size of the user.

When detecting that the user terminal 110 is within a predetermined distance from the determined wearable device 1820, the server 140 may transmit, to the determined wearable device 1820, a control signal to cause the determined wearable device 1820 to provide a notification to the user. For example, the server 140 may identify the location of the user terminal 110 by using global positioning system (GPS) information of the user terminal 110, information of the access point to which the user terminal 110 is connected, and the like. Through this location detection, the server 140 may determine whether the user terminal 110 is within a predetermined distance from the determined wearable device 1820. When the user terminal 110 is within the predetermined distance from the determined wearable device 1820, the server 140 may transmit, to the determined wearable device 1820, a control signal to cause the determined wearable device 1820 to provide a notification (e.g., a motion and/or lighting) to the user.

For example, in the example illustrated in FIG. 18D, when detecting that the user terminal 110 is within the predetermined distance from the wearable device 1820, the server 140 may transmit, to the wearable device 1820, a control signal to provide a notification to the user. When determining that the wearable device 1820 is stably fixed to a cradle, the wearable device 1820 may perform a motion and/or lighting. When determining that the wearable device 1820 is stably fixed to the cradle, the wearable device 1820 may perform sound output and/or lighting. A wearable device 1830 located in the vicinity of the wearable device 1820 may not react even when the user terminal 110 approaches the wearable device 1830. Accordingly, the user may easily recognize that the wearable device 1820 that reacts when the user terminal 110 approaches is a wearable device used for performing an exercise.

According to an embodiment, when the user terminal 110 is within the predetermined distance from the wearable device 1820, the server 140 may transmit, to the wearable device 1820, a control signal instructing to provide a notification to the user. The wearable device 1820 may detect, through a beacon sensor or near field communication (NFC), that the user terminal 110 is within the predetermined distance from the wearable device 1820. The wearable device 1820 may provide a notification to the user when the user terminal 110 is within the predetermined distance from the wearable device 1820.

The embodiments described with reference to FIGS. 1 to 17 may apply to the embodiments of FIGS. 18A, 18B, 18C, and 18D.

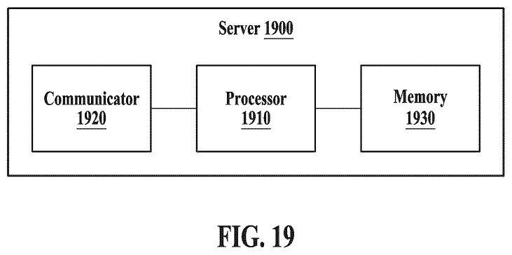

FIG. 19 is a block diagram illustrating an example of a server according to an embodiment.

Referring to FIG. 19, a server 1900 (e.g., the server 140) may include a processor 1910, a communicator 1920, and a memory 1930.

The communicator 1920 may perform wired or wireless communication.

The processor 1910 may store the size of each of the plurality of wearable devices 410 in the memory 1930.

The processor 1910 may store, in the memory 1930, information about a place (e.g., a fitness center) in which a user primarily performs an exercise.

The processor 1910 may receive, from the user terminal 110, condition information including a wearable device search condition through the communicator 1920.

The processor 1910 may receive, from each of the plurality of wearable devices 410, device information of each of the plurality of wearable devices 410 through the communicator 1920.

The processor 1910 may select at least one wearable device (e.g., the wearable device A 411 of FIG. 5, etc.) for a user among the wearable devices 410 based on the received state information and received condition information.

The processor 1910 may transmit, to the selected wearable device through the communicator 1920, a control signal to provide the user with a notification indicating that the selected wearable device matches the wearable device search condition. The control signal may command the selected wearable device to perform at least one of driving an actuator or lighting light sources.

When receiving the control signal from the server 1900, the selected wearable device (e.g., the wearable device A 411 of FIG. 5) may determine whether the selected wearable device is fixed to a cradle. When determining that the selected wearable device is fixed to the cradle, the selected wearable device may perform, based on the control signal, at least one of driving the actuator of the selected wearable device or lighting the light sources of the selected wearable device. When determining that the selected wearable device is not fixed to the cradle, the selected wearable device may perform at least one of lighting the light sources or sound output.

In an embodiment, the received condition information may include an exercise type and an exercise duration input by the user, and the received device information may include the charge state of each of the wearable devices 410. Based on the input exercise type, the input exercise duration, and the charge state of each of the wearable devices 410, the processor 1910 may select a wearable device that may be used for the exercise time or more among the wearable devices 410. For example, using the wearable device power consumption set for the input exercise type and the charge state of each of the wearable devices 410, the processor 1910 may calculate the estimated usable time of each of the wearable devices 410. The processor 1910 may select a wearable device (e.g., the wearable devices 612 and 613 of FIG. 6) having an estimated usable time greater than or equal to the exercise duration.

In an embodiment, the received condition information may include at least one of a size or a charge rate input by the user, and each piece of received device information may include the size of each of the wearable devices 410. The processor 1910 may select a wearable device (e.g., the wearable device 611 of FIG. 8) having the input size among the wearable devices 410 or may select a wearable device (e.g., the wearable devices 611 and 612 of FIG. 7) having a charge state less than or equal to the input charge rate among the wearable devices 410.

In an embodiment, based on the exercise type, the exercise duration, the size input by the user, the charge state of each of the wearable devices 410, and the size of each of the wearable devices 410, the processor 1910 may select a first wearable device that satisfies a plurality of standards and may select a second wearable device that satisfies some of the standards. The processor 1910 may transmit, to the selected first wearable device through the communicator 1920, a first control signal to notify the user that the selected first wearable device satisfies the plurality of standards. The processor 1910 may transmit, to the selected second wearable device through the communicator 1920, a second control signal to notify the user that the selected second wearable device satisfies some of the plurality of standards.

The plurality of standards may include, for example, a first standard that is satisfied when the size of a predetermined wearable device matches the size input by the user and a second standard that is satisfied when the estimated usable time calculated using the wearable device power consumption set for the exercise type and the charge state of each of the wearable devices 410 is greater than or equal to the exercise duration.

When receiving a first control signal from the server 1900, the selected first wearable device (e.g., the wearable device 611 described with reference to FIG. 10) may cause first and second frames (e.g., the first and second connection frames) of the selected first wearable device to move by generating a torque through an actuator and may perform first lighting through light sources of the selected first wearable device.

When receiving a second control signal from the server 1900, the selected second wearable device (e.g., the wearable device 612 described with reference to FIG. 10) may allow the first and second frames (e.g., the first and second connection frames) of the selected second wearable device to stay separated by a predetermined angle (e.g., twice the first angle) and may perform second lighting through the light sources of the selected second wearable device.

In an embodiment, the processor 1910 may receive, from the administrator terminal 710 through the communicator 1920, a request for checking a state selected by an administrator among states (e.g., a regular maintenance state, a charge state (a low battery state) or a state for a pairing record (or a wireless communication connection record)) of a target wearable device of the plurality of wearable devices 410. Based on the received check request, the processor 1910 may transmit, to the target wearable device, a control signal to allow the target wearable device to notify the administrator of a state in which the target wearable device is selected.

When receiving, from the server 1900, a control signal to notify the administrator that the regular maintenance state selected by the administrator among the states is an unexecuted state, the target wearable device may perform a motion corresponding to the unexecuted state and third lighting.

The motion corresponding to the unexecuted state and the third lighting may be performed for a first time (e.g., 30 seconds) when the timepoint of the request for checking the selected regular maintenance state is within a predetermined period after the regular maintenance date of the target wearable device elapses. The third lighting and the motion corresponding to the unexecuted state may be performed for a second time (e.g., 1 minute), which is greater than the first time, when the timepoint of the request for checking the regular maintenance state is a predetermined duration after the regular maintenance elapses. The first time and the second time are not limited to the examples described above.

When receiving, from the server 1900, a control signal to notify the administrator of a low battery state selected by the manager among the states, the target wearable device may check a charge state. When the checked charge state is a low battery state, the target wearable device may perform fourth lighting and a motion corresponding to the low battery state.

The fourth lighting and the motion corresponding to the low battery state may be performed for a third time (e.g., 30 seconds) when the charge state of the target wearable device is less than or equal to a first level (e.g., 10%). The fourth lighting and the motion corresponding to the low battery state may be performed for a fourth time (e.g., 1 minute) when the charge state of the target wearable device is less than or equal to a second level (e.g., 5%), which is less than the first level. The third time, the fourth time, the first level, and the second level are not limited to the examples described above.

When receiving, from the server 1900, a control signal to notify the administrator of the state of a wireless communication connection record selected by the administrator among the states, the target wearable device may check the wireless communication connection record. When there is no wireless communication connection record, the target wearable device may perform fifth lighting and a motion corresponding to a state in which there is no wireless communication connection record.

The fifth lighting and the motion corresponding to the state in which there is no wireless communication connection record may be performed for a fifth time (e.g., 30 seconds) when there is no wireless communication connection record of the target wearable device during the first period. The fifth lighting and the motion corresponding to the state in which there is no wireless communication connection record may be performed during a sixth time (e.g., 1 minute) when there is no wireless communication connection record during the second period. The fifth lighting and the motion corresponding to the state in which there is no wireless communication connection record may be performed for a seventh period (e.g., 2 minutes) when there is no wireless communication connection record exceeding the second period. The fifth time, the sixth time, and the seventh time are not limited to the examples described above.

In an embodiment, the processor 1910 may receive, from the user terminal 110 through the communicator 1920, a message indicating that the user terminal 110 is in wireless communication connection with one of the wearable devices providing a notification. The processor 1910 may transmit, through the communicator 1920, a control signal to prevent or reduce chances of notifications from being provided to any wearable device other than the wearable device that is in wireless communication connection with the user terminal 110 among the wearable devices providing notifications.

In an embodiment, the processor 1910 may determine a recommended wearable device recommended to the user from the selected wearable device. The processor 1910 may transmit, to the user terminal 110, information about a recommended wearable device through the communicator 1920. The processor 1910 may receive, from the user terminal 110 through the communicator 1920, a message indicating that a wireless communication connection with the recommended wearable device is in progress. The processor 1910 may transmit, to the user terminal 110 through the communicator 1920, a message indicating that the recommended wearable device is in wireless communication connection with another user terminal. When there is another recommended wearable device for the user, the processor 1910 may transmit, to the user terminal 110, information about another recommended wearable device.

In an embodiment, the processor 1910 may determine a first recommended wearable device recommended to the user among the selected wearable devices. The processor 1910 may transmit, to the user terminal 110 through the communicator 1920, information about the first recommended wearable device. The processor 1910 may transmit, to the first recommended wearable device through the communicator 1920, a control signal instructing to perform a first motion. When receiving, from another user terminal through the communicator 1920, condition information including a wearable device search condition of another user, the processor 1910 may determine, among the plurality of wearable devices 410, a second recommended wearable device recommended to another user in a way that the second recommended wearable device is not the same as the first recommended wearable device. The processor 1910 may transmit, to another user terminal through the communicator 1920, information about the second recommended wearable device. The processor 1910 may transmit, to the second recommended wearable device through the communicator 1920, a control signal instructing to perform a second motion, which is different from the first motion.

In an embodiment, the processor 1910 may receive, from the user terminal 110 through the communicator 1920, a reservation request for scheduling an exercise reservation. The processor 1910 may transmit, to the administrator terminal 1810, a message indicating that there is a reservation request.

In an embodiment, the processor 1910 may receive, from the user terminal 110 through the communicator 1920, a reservation request for scheduling an exercise reservation. When detecting that the user terminal 110 is within a predetermined distance from a wearable device (e.g., the wearable device 1820) that is determined to satisfy a condition (e.g., size) of the user and a charge state, the processor 1910 may transmit, to the determined wearable device through the communicator 1920, a control signal to cause the determined wearable device to provide a notification to the user.

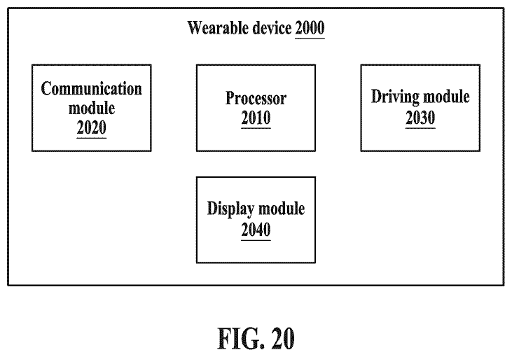

FIG. 20 is a block diagram illustrating an example of a wearable device according to an embodiment.

Referring to FIG. 20, a wearable device 2000 (e.g., the wearable devices 120, 200, 300, and 300-1) may include a processor 2010 (e.g., the processor 310), a communication module 2020 (e.g., the communication module 390, comprising communication circuitry), a driving module 2030 (e.g., the driving module 30 comprising a motor and/or circuitry), and a display module 2040. The display module 2040 may include a lighting unit 60.

The communication module 2020 may communicate with a server 140, 1900.

The driving module 2030 may include an actuator that generates a torque.

The processor 2010, comprising processing circuitry, may receive, from the server 140, 1900 through the communication module 2020, a control signal to provide a user with a notification indicating that the wearable device 2000 matches a wearable device search condition of the user.

The processor 2010 may cause at least one of driving the actuator or providing visual feedback (e.g., lighting) from the display module (e.g., light sources) to be performed based on the received control signal.

In an embodiment, the processor 2010 may determine, through a sensor, whether the wearable device 2000 is fixed to a cradle. When determining that the wearable device 2000 is fixed to the cradle, the processor 2010 may cause at least one of driving the actuator or lighting light sources to be performed. When determining that the wearable device 2000 is not fixed to the cradle, the processor 2010 may cause at least one of lighting and sound output to be performed.

In an embodiment, the processor 2010 may receive, from the server 140, 1900 through the communication module 2020, a first control signal to notify the user that the wearable device 2000 satisfies a plurality of standards. The processor 2010 may cause first and second frames (e.g., first and second connection frames) of the wearable device 2000 to move by generating a torque through the actuator based on the received first control signal and cause light sources to perform first lighting.

The processor 2010 may receive, from the server 140, 1900 through the communication module 2020, a second control signal to notify the user that the wearable device 2000 satisfies some of the plurality of standards. The processor 2010 may allow the first and second frames (e.g., the first and second connection frames) of the wearable device 2000 to stay separated by a predetermined angle based on the received second control signal and cause the light sources to perform second lighting.

The embodiments described with reference to FIGS. 1 to 19 may apply to the wearable device 2000 of FIG. 20.

The embodiments described herein may be implemented using a hardware component, a software component, and/or a combination thereof. A processing device may be implemented using one or more general-purpose or special-purpose computers, such as, for example, a processor, a controller and an arithmetic logic unit (ALU), a digital signal processor (DSP), a microcomputer, a field-programmable gate array (FPGA), a programmable logic unit (PLU), a microprocessor, or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is singular; however, one of ordinary skill in the art will appreciate that a processing device may include multiple processing elements and multiple types of processing elements. For example, the processing device may include a plurality of processors, or a single processor and a single controller. In addition, different processing configurations are possible, such as parallel processors.

The software may include a computer program, a piece of code, an instruction, or one or more combinations thereof, to independently or collectively instruct or configure the processing device to operate as desired. Software and/or data may be embodied permanently or temporarily in any type of machine, component, physical or virtual equipment, computer storage medium or device, or in a propagated signal wave capable of providing instructions or data to or being interpreted by the processing device. The software also may be distributed over network-coupled computer systems so that the software is stored and executed in a distributed fashion. The software and data may be stored by one or more non-transitory computer-readable recording mediums.

The methods according to the above-described embodiments may be recorded in non-transitory computer-readable media including program instructions to implement various operations of the above-described embodiments. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of non-transitory computer-readable media include magnetic media such as hard disks, floppy disks, and magnetic tape; optical media such as CD-ROM discs and DVDs; magneto-optical media such as optical discs; and hardware devices that are specially configured to store and perform program instructions, such as read-only memory (ROM), random access memory (RAM), flash memory, and the like. Examples of program instructions include both machine code, such as produced by a compiler, and files containing higher-level code that may be executed by the computer using an interpreter.

The above-described hardware devices may be configured to act as one or more software modules in order to perform the operations of the above-described embodiments, or vice versa.

As described above, although the embodiments have been described with reference to the limited drawings, one of ordinary skill in the art may apply various technical modifications and variations based thereon. For example, suitable results may be achieved if the described techniques are performed in a different order, and/or if components in a described system, architecture, device, or circuit are combined in a different manner, or replaced or supplemented by other components or their equivalents.

Therefore, other implementations, other embodiments, and equivalents of the claims are within the scope of the following claims.

1. A wearable device control method performed by a server, the wearable device control method comprising:
receiving, from a user terminal, condition information comprising a wearable device search condition;
receiving, from each of a plurality of wearable devices, device information regarding each of the wearable devices;
based on the received state information and the received condition information, selecting, from among the wearable devices, at least one wearable device for a user; and
transmitting, to the selected wearable device, a control signal to provide the user with a notification indicating that the selected wearable device matches the wearable device search condition.

2. The wearable device control method of claim 1, wherein the control signal instructs the selected wearable device to perform at least one of driving an actuator or lighting light sources.

3. The wearable device control method of claim 1, wherein
the received condition information comprises an exercise type and an exercise duration input by the user, and the received device information comprises a charge state of each of the wearable devices, and
the selecting of the at least one wearable device comprises selecting, from among the wearable devices, based on the input exercise type, the input exercise duration, and the charge state of each of the plurality of wearable devices, a wearable device usable for an exercise duration and/or longer.

4. The wearable device control method of claim 3, wherein the selecting of the wearable device available for the exercise duration and/or longer comprises:
calculating, using wearable device power consumption set for the input exercise type and the charge state of each of the plurality of wearable devices, an estimated usable time of each of the wearable devices; and
selecting a wearable device having an estimated usable time greater than or equal to the exercise duration.

5. The wearable device control method of claim 1, wherein
the received condition information comprises at least one of a size or a charge rate input by the user, the received device information comprises a size of each of the wearable devices, and the selecting of the wearable device comprises selecting a wearable device having the input size among the wearable devices and/or selecting a wearable device having a charge state less than or equal to the input charge rate among the wearable devices, and/or
the received condition information comprises the exercise type, the exercise duration, and the size input by the user, the received device information comprises a charge state of each of the wearable devices, the selecting of the wearable device comprises selecting, based on the input exercise type, the input exercise duration, the input size, the charge state of each of the wearable devices, and the size of each of the wearable devices, a first wearable device satisfying a plurality of standards and selecting a second wearable device satisfying some of the standards, and the transmitting of the control signal comprises transmitting, to the selected first wearable device, a first control signal to notify a user that the selected first wearable device satisfies the standards and transmitting, to the selected second wearable device, a second control signal to notify the user that the selected second wearable device satisfies some of the standards.

6. The wearable device control method of claim 1, wherein the selected wearable device is configured to determine whether the selected wearable device is fixed to a cradle when receiving the control signal from the server, perform, based on the control signal, driving an actuator of the selected wearable device or lighting light sources of the selected wearable device when determining that the selected wearable device is fixed to the cradle, and perform at least one of lighting of the light sources or sound output when determining that the selected wearable device is not fixed to the cradle.

7. The wearable device control method of claim 5, wherein
the plurality of standards comprises a first standard satisfied when a size of a predetermined wearable device matches the input size and a second standard satisfied when the estimated usable time, which is calculated using the wearable device power consumption set for the input exercise type and the charge state of each of the wearable devices, is greater than or equal to the input exercise duration, and/or
the selected first wearable device is configured to cause a first frame and a second frame of the selected first wearable device to move by generating a torque through an actuator when receiving the first control signal, perform first lighting through light sources of the selected first wearable device, and the selected second wearable device is configured to allow a first frame and a second frame of the selected second wearable device to stay separated by a predetermined angle when receiving the second control signal, and perform second lighting through light sources of the selected second wearable device.

8. The wearable device control method of claim 1, further comprising:
receiving, from an administrator terminal, a request for checking a state selected by an administrator among states of a target wearable device of the plurality of wearable devices; and
based on the received request for checking the state, transmitting, to the target wearable device, a control signal to allow the target wearable device to notify the administrator of the selected state of the target wearable device.

9. The wearable device control method of claim 8, wherein the target wearable device is configured to:
when receiving, from the server, a control signal to notify the administrator that a regular maintenance state selected by the administrator is an unexecuted state among the states, perform third lighting and a motion corresponding to the unexecuted state;
when receiving, from the server, a control signal to notify the administrator of a low battery state selected by the administrator among the states, check a charge state and perform fourth lighting and a motion corresponding to the low battery state; and
when receiving, from the server, a control signal to notify the administrator of a state of a wireless communication connection record selected by the administrator among the states, check the wireless communication connection record and when the wireless communication connection record is absent, perform fifth lighting and a motion corresponding to a state in which the wireless communication connection record is absent.

10. The wearable device control method of claim 9, wherein
the third lighting and the motion corresponding to the unexecuted state are performed for a first time when a timepoint of a request for checking the selected regular maintenance state is within a predetermined period after a regular maintenance date of the target wearable device elapses and performed during a second time, which is longer than the first time, when the timepoint of the request for checking the selected regular maintenance state exceeds the predetermined period,
the fourth lighting and the motion corresponding to the low battery state are performed for a third time when a charge state of the target wearable device is less than or equal to a first level, performed for a fourth time when the charge rate of the target wearable device is less than or equal to a second level, which is lower than the first level, and
the fifth lighting and the motion corresponding to the state in which the wireless communication connection record is absent are performed for a fifth time when a wireless communication connection record of the target wearable device is absent during a first period, performed for a sixth time when the wireless communication connection record is absent during a second period, and performed for a seventh time when the wireless communication connection record is absent during a period greater than the second period.

11. The wearable device control method of claim 1, further comprising:
receiving, from the user terminal, a message indicating that one of wearable devices providing the notification and the user terminal perform a wireless communication connection and transmitting, to rest of the wearable devices providing the notification except a wearable device performing the wireless communication connection with the user terminal, a control signal to prevent or reduce chances of the notification from being provided; and/or
determining a recommended wearable device recommended to the user from the selected wearable device, transmitting, to the user terminal, information about the recommended wearable device, receiving, from the user terminal, a message indicating that a wireless communication connection with the recommended wearable device is in progress, transmitting, to the user terminal, a message indicating that the recommended wearable device is in wireless communication connection with another user terminal, and when there is another recommended wearable device for the user, transmitting, to the user terminal, information about the another recommended wearable device.

12. The wearable device control method of claim 1, further comprising:
determining a first recommended wearable device recommended to the user from the selected wearable device, transmitting, to the user terminal, information about the first recommended wearable device, transmitting, to the first recommended wearable device, a control signal instructing to perform a first motion, when condition information comprising a wearable device search condition of another user is received from another user terminal, determining, among the plurality of wearable devices, a second wearable device recommended to the another user in a way that the second recommended wearable device is different from the first recommended wearable device, transmitting, to the another user, information about the second recommended wearable device, and transmitting, to the second recommended wearable device, a control signal instructing to perform a second motion different from the first motion;
receiving, from the user terminal, a reservation request for scheduling an exercise reservation and transmitting, to an administrator terminal, a message indicating that the reservation request is present; and/or
when it is detected that the user terminal is within a predetermined distance from a wearable device determined to satisfy a condition of the user and a charge state, transmitting, to the determined wearable device, a control signal to allow the determined wearable device to provide a notification to the user.

13. A wearable device comprising:
a communication module, comprising communication circuitry, configured to communicate with a server;
a driving module comprising an actuator, comprising a motor and/or circuitry, configured to generate a torque; and
at least one processor, comprising processing circuitry, individually and/or collectively configured to receive, from the server through the communication module, a control signal to provide a user with a notification indicating that the wearable device matches a wearable device search condition of the user, and perform, based on the received control signal, at least one of driving the actuator or providing visual feedback.

14. The wearable device of claim 13, wherein
the at least one processor is individually and/or collectively configured to determine, through a sensor, whether the wearable device is fixed to a cradle, when determining that the wearable device is fixed to the cradle, perform at least one of the driving the actuator or lighting a plurality of light sources of the wearable device, and when determining that the wearable device is not fixed to the cradle, perform at least one of the lighting or sound output.

15. The wearable device of claim 13, wherein
the at least one processor is individually and/or collectively configured to:
when receiving, from the server through the communication module, a first control signal to notify that the wearable device satisfies a plurality of standards, based on the received first control signal, cause a first frame and a second frame of the wearable device to move by generating a torque through the actuator and cause a plurality of light sources of the wearable device to perform first lighting; and
when receiving, from the server through the communication module, a second control signal to notify the user that the wearable device satisfies some of the plurality of standards, based on the received second control signal, allow the first frame and the second frame of the wearable device to stay separated by a predetermined angle and cause the light sources to perform second lighting.
